(19) United States
(12) Patent Application Publication
(10) Pub. No.: US 2022/0313105 A1
(43) Pub. Date: Oct. 6, 2022

(54) MEDICAL DEVICE AND METHOD FOR IMPEDANCE MONITORING

(71) Applicant: Medtronic, Inc., Minneapolis, MN (US)

(72) Inventors: Vladimir P. NIKOLSKI, Blaine, MN (US); Vinod SHARMA, Maple Grove, MN (US)

(21) Appl. No.: 17/686,292

(22) Filed: Mar. 3, 2022

Related U.S. Application Data
(60) Provisional application No. 63/170,015, filed on Apr. 2, 2021.

(51) Int. Cl. A61B 5/0537; A61B 5/00; A61B 5/25; G16H 50/30

(52) U.S. Cl. CPC A61B 5/0537; A61B 5/74; A61B 5/686; A61B 5/0015; A61B 5/25; G16H 50/30

(57) ABSTRACT

A medical device is configured to obtain impedance measurements from each one of multiple impedance measurement electrode vectors and determine an estimate of impedance of body tissue or a body cavity, e.g., a thoracic impedance estimate, by computing an impedance of a circuit model of impedance using the multiple impedance measurements. The medical device may be configured to determine that the impedance estimate meets fluid condition detection criteria and detect a fluid status condition in response to the impedance estimate meeting the fluid condition detection criteria.

### REFERENCE TO RELATED APPLICATIONS

This application claims the benefit of provisional U.S. Patent Application No. 63/170,015, filed on Apr. 2, 2021, incorporated herein by reference in its entirety.

### TECHNICAL FIELD

The disclosure relates generally to a medical device and method for monitoring impedance changes associated with changes in fluid content of a tissue or body region. day

### BACKGROUND

Medical devices may sense a variety of signals for monitoring a patient condition. Such devices may be external (e.g., bedside or wearable monitors) or implantable monitors, which may include therapy delivery capabilities in some cases, such as cardiac pacing or drug delivery. Some medical devices have been used, or proposed for use, in monitoring heart failure to detect worsening of a heart failure condition by monitoring thoracic impedance. Changes in tissue impedance may provide a good indication of the level of edema in patients. While edema is a sign of many other conditions it is also a sign of worsening heart failure.

In the early stages of heart failure, compensatory mechanisms occur in response to the heart's inability to pump a sufficient amount of blood. One compensatory response is an increase in filling pressure of the heart. The increased filling pressure increases the volume of blood in the heart, allowing the heart to eject a larger volume of blood on each heartbeat. Increased filling pressure and other compensatory mechanisms can initially occur without overt heart failure symptoms. The mechanisms that initially compensate for insufficient cardiac output, however, lead to heart failure decompensation as the heart continues to weaken. The weakened heart can no longer pump effectively causing increased filling pressure to lead to chest congestion (thoracic edema) and heart dilation, which further compromises the heart's pumping function. The patient begins the “vicious cycle” of heart failure.

Heart chamber dilation, increased pulmonary blood volume, and fluid retention in the lungs all contribute to a decrease in thoracic impedance. Generally, the first indication that a physician would have of the occurrence of edema in a patient is not until it becomes a physical manifestation with swelling or breathing difficulties which generally leads to hospitalization. Typically, therapy for a patient hospitalized for acute decompensated heart failure includes early introduction of intravenous infusion of diuretics or vasodilators to clear excess fluid retained by the patient. Early detection of edema, before serious symptoms arise, enables earlier management and intervention, e.g., through pharmaceutical agents, cardiac resynchronization therapy (CRT), or other therapies, which may circumvent more serious patient symptoms and the reduce likelihood of hospitalizations due to edema.

### SUMMARY

In general, the disclosure is directed to a device and method for determining a thoracic impedance estimate based on multiple impedance measurements. The thoracic impedance estimate may be used in monitoring for detecting a fluid status condition of the patient, e.g., edema associated with worsening heart failure or dehydration associated with over-diruesis. In some examples, the techniques disclosed herein enable thoracic impedance monitoring to be performed using extra-cardiac electrodes, which may be implanted subcutaneously, submuscularly, substernally or transvenously but outside the heart, as examples. A medical device operating according to the disclosed techniques obtains an impedance measurement from each one of multiple impedance measurement electrode vectors.

Using the impedance measurements, the medical device may compute the thoracic impedance estimate according to a circuit model of thoracic impedance that includes multiple impedance elements, where one or more of the impedance measurements represents a series or parallel combination of at least one of the circuit model impedance elements. In this way, an impedance of the circuit model of thoracic impedance may be computed and used as a thoracic impedance measurement that is closely correlated to an actual intrathoracic impedance measurement. The thoracic impedance estimate may be compared to fluid status condition criteria for detecting a condition, e.g., edema or over-diuresis, which may warrant a therapy response by a medical device or other medical attention.

In one example, the disclosure provides a medical device including an impedance measurement circuit configured to obtain an impedance measurement between each one of multiple impedance measurement electrode vectors and a control circuit configured to determine a thoracic impedance estimate by computing an impedance of a circuit model of thoracic impedance using the impedance measurements. The circuit model of thoracic impedance includes multiple impedance elements extending between at least three terminals. The control circuit may determine that the thoracic impedance estimate meets fluid status condition criteria and detect a fluid status condition in response to the thoracic impedance estimate meeting the fluid status condition criteria. The control circuit may generate an output in response to detecting the fluid status condition. The medical device may include a memory configured to store data relating to the thoracic impedance estimate in response to the generated output.

In another example, the disclosure provides a method including obtaining an impedance measurement between each one of multiple impedance measurement electrode vectors and determining a thoracic impedance estimate by computing an impedance of a circuit model of thoracic impedance using the impedance measurements. The circuit model of thoracic impedance includes multiple impedance elements extending between at least three terminals. The method may include determining that the thoracic impedance estimate meets fluid status condition criteria and detecting a fluid status condition in response to the thoracic impedance estimate meeting the fluid status condition criteria. The method may further include generating an output in response to detecting the fluid status condition and storing data relating to the thoracic impedance estimate in response to the generated output.

In another example, the disclosure provides a non-transitory computer-readable medium storing a set of instructions which, when executed by a control circuit of a medical device cause the device to obtain an impedance measurement between each of a plurality of impedance measurement electrode vectors and determine a thoracic impedance estimate by computing an impedance of a circuit model of thoracic impedance using the impedance measurements. The circuit model of thoracic impedance includes a plurality of impedance elements extending between at least three terminals. The instructions may further cause the device to determine that the thoracic impedance estimate meets fluid status condition criteria and detect a fluid status condition in response to the thoracic impedance estimate meeting the fluid status condition criteria. The instructions may cause the device to generate an output in response to detecting the fluid status condition and store data relating to the thoracic impedance estimate in response to the generated output.

Further disclosed herein is the subject matter of the following examples: Example 1. A medical device including an impedance measurement circuit configured to obtain an impedance measurement between each of a plurality of impedance measurement electrode vectors and a control circuit configured to determine a thoracic impedance estimate by computing an impedance of a circuit model of thoracic impedance using the impedance measurements. The circuit model of thoracic impedance can include a plurality of impedance elements extending between at least three terminals. The control circuit can be further configured to determine that the thoracic impedance estimate meets fluid status condition criteria, detect a fluid status condition in response to the thoracic impedance estimate meeting the fluid status condition criteria, and generate an output in response to detecting the fluid status condition. The medical device may include a memory configured to store data relating to the thoracic impedance estimate in response to the generated output.

Example 2. The medical device of example 1 wherein the control circuit is further configured to determine the thoracic impedance estimate by computing an equivalent impedance of the plurality of impedance elements of the circuit model.

Example 3. The medical device of any of examples 1-2 wherein the impedance measurement circuit is further configured to obtain each of the impedance measurements by determining an impedance measurement corresponding to a combination of at least two of the impedance elements of the circuit model.

Example 4. The medical device of any of examples 1-3 wherein the control circuit is further configured to compute the equivalent impedance of the circuit model from the impedance measurements by computing an equivalent impedance of a wye circuit model comprising three impedance elements, wherein at least one of the impedance measurements corresponds to a series combination of at least two of the three impedance elements of the wye circuit model.

Example 5. The medical device of example 4 wherein the impedance measurement circuit is further configured to obtain at least one of the impedance measurements corresponding to a first impedance element of the three impedance elements of the wye circuit model in series with a parallel combination of a second impedance element and a third impedance element of the three impedance elements of the wye circuit model.

Example 6. The medical device of any of examples 1-5 further comprising a housing enclosing the impedance measurement circuit and the control circuit and wherein the impedance measurement circuit is further configured to obtain the impedance measurements by determining at least a first impedance measurement from a first impedance measurement electrode vector of the plurality of impedance measurement electrode vectors, the first impedance measurement electrode vector being between a first electrode and a second electrode when the first and second electrodes are coupled to the impedance measurement circuit, and a second impedance measurement from a second impedance measurement electrode vector of the plurality of impedance measurement electrode vectors, the second impedance measurement electrode vector being between the first electrode and the housing. The control circuit may be further configured to determine the thoracic impedance estimate by determining an equivalent impedance of a three terminal circuit model using the impedance measurements, wherein the first impedance measurement corresponds to a series combination of a first impedance element and a second impedance element of the three terminal circuit model and the second impedance measurement corresponds to a series combination of the first impedance element and a third impedance element of the three terminal circuit model.

Example 7. The medical device of example 6 wherein the impedance measurement circuit is further configured to obtain the impedance measurements by obtaining a third impedance measurement from a third impedance measurement electrode vector of the plurality of impedance measurement electrode vectors. The third impedance measurement electrode vector can be between the first electrode and a combination of the second electrode and the housing.

Example 8. The medical device of any of examples 1-5 wherein the control circuit is further configured to determine the thoracic impedance estimate by determining an impedance of a single impedance element of the circuit model of thoracic impedance using the impedance measurements.

Example 9. The medical device of any of examples 1-8 wherein the control circuit is further configured to determine that the thoracic impedance estimate meets the fluid status criteria by determining that the thoracic impedance estimate is outside a normal impedance range and detecting a fluid status condition in response to the thoracic impedance estimate being outside the normal impedance range.

Example 10. The medical device of any of examples 1-9 wherein the control circuit is further configured to determine that the thoracic impedance estimate meets the fluid status criteria by establishing a baseline thoracic impedance, determining a fluid status index by determining a cumulative sum of differences between a plurality of consecutively determined thoracic impedance estimates and the baseline thoracic impedance, determining that the fluid status index crosses a threshold and determining that the fluid status criteria are met in response to the fluid status index crossing the threshold.

Example 11. The medical device of any of claims 1-10 wherein the control circuit is further configured to determine the thoracic impedance estimate by computing an impedance of one of a star circuit model of the plurality of impedance elements or a mesh circuit model of the plurality of impedance elements.

Example 12. The medical device of any of claims 1-11 further comprising a telemetry circuit configured to transmit a fluid status notification signal in response to the generated output.

Example 13. The medical device of any of examples 1-12 wherein the impedance measurement circuit is further configured to obtain the impedance measurements from a plurality of impedance measurement electrode vectors comprising at least two electrodes carried by an extra-cardiac, implantable lead.

Example 14. The medical device of any of claims 1-13 further comprising a telemetry circuit and the control circuit is further configured to receive a user selection signal via the telemetry circuit indicating at least one of a selectable impedance measurement electrode included in the plurality of the impedance measurement electrode vectors, the circuit model of thoracic impedance, or one of the plurality of impedance elements of the circuit model. The control circuit can be configured to determine the thoracic impedance estimate by computing the impedance of the circuit model of thoracic impedance according to the user selection signal.

Example 15. A method comprising obtaining an impedance measurement between each of a plurality of impedance measurement electrode vectors and determining a thoracic impedance estimate by computing an impedance of a circuit model of thoracic impedance using the impedance measurements. The circuit model of thoracic impedance includes a plurality of impedance elements extending between at least three terminals. The method may further include determining that the thoracic impedance estimate meets fluid status condition criteria, detecting a fluid status condition in response to the thoracic impedance estimate meeting the fluid status condition criteria, and generating an output in response to detecting the fluid status condition. The method may further include storing data relating to the thoracic impedance estimate in response to the generated output.

Example 16. The method of example 15 wherein determining the thoracic impedance estimate further comprises computing an equivalent impedance of the plurality of impedance elements of the circuit model.

Example 17. The method of any of examples 15-16 wherein obtaining each of the impedance measurements further comprises determining an impedance measurement corresponding to a combination of at least two of the impedance elements of the circuit model.

Example 18. The method of any of examples 15-17, wherein computing the equivalent impedance of the circuit model using the impedance measurements further comprises computing an equivalent impedance of a wye circuit model comprising three impedance elements, wherein at least one of the impedance measurements corresponds to a series combination of at least two of the three impedance elements of the wye circuit model.

Example 19. The method of example 18 wherein obtaining the impedance measurements further comprises obtaining an impedance measurement corresponding to a first impedance element of the three impedance elements of the wye circuit model in series with a parallel combination of a second impedance element and a third impedance element of the three impedance elements of the wye circuit model.

Example 20. The method of any of examples 15-19 further comprising obtaining the impedance measurements by determining at least a first impedance measurement from a first impedance measurement electrode vector of the plurality of impedance measurement electrode vectors, the first impedance measurement electrode vector being between a first electrode and a second electrode, and a second impedance measurement from a second impedance measurement electrode vector of the plurality of impedance measurement electrode vectors, the second impedance measurement electrode vector being between the first electrode and a housing of the medical device. The method may further include determining the thoracic impedance estimate by determining an equivalent impedance of a three terminal circuit model using the impedance measurements, wherein the first impedance measurement corresponds to a series combination of a first impedance element and a second impedance element of the three terminal circuit model and the second impedance measurement corresponds to a series combination of the first impedance element and a third impedance element of the three terminal circuit model.

Example 21. The method of example 20 wherein obtaining the impedance measurements further comprises obtaining a third impedance measurement from a third impedance measurement electrode vector of the plurality of impedance measurement electrode vectors. The third impedance measurement electrode vector can be between the first electrode and a combination of the second electrode and the housing.

Example 22. The method of any of examples 15-19 further comprising determining the thoracic impedance estimate by determining an impedance of a single impedance element of the circuit model of thoracic impedance using the impedance measurements.

Example 23. The method of any of examples 15-22 wherein determining that the thoracic impedance estimate meets the fluid status criteria further comprises determining that the thoracic impedance estimate is outside a normal impedance range and detecting a fluid status condition in response to the thoracic impedance estimate being outside the normal impedance range.

Example 24. The method of any of examples 15-23 wherein determining that the thoracic impedance estimate meets the fluid status criteria further comprises establishing a baseline thoracic impedance, determining a fluid status index by determining a cumulative sum of differences between a plurality of consecutively determined thoracic impedance estimates and the baseline thoracic impedance, determining that the fluid status index crosses a threshold, and determining that the fluid status criteria are met in response to the fluid status index crossing the threshold.

Example 25. The method of any of examples 15-24 wherein determining the thoracic impedance estimate further comprises computing an impedance of one of a star circuit model of the plurality of impedance elements or a mesh circuit model of the plurality of impedance elements.

Example 26. The method of any of examples 15-25 further comprising transmitting a fluid status notification signal in response to the generated output.

Example 27. The method of any of examples 15-26 further comprising receiving a user selection signal indicating at least one of a selectable impedance measurement electrode included in the plurality of the impedance measurement electrode vectors, the circuit model of thoracic impedance, or one of the plurality of impedance elements of the circuit model. The method can further include determining the thoracic impedance estimate by computing the impedance of the circuit model of thoracic impedance according to the user selection signal.

Example 28. A non-transitory computer-readable medium storing a set of instructions which, when executed by a control circuit of a medical device, cause the device to obtain an impedance measurement between each of a plurality of impedance measurement electrode vectors and determine a thoracic impedance estimate by computing an impedance of a circuit model of thoracic impedance using the impedance measurements. The circuit model of thoracic impedance can include a plurality of impedance elements extending between at least three terminals. The control circuit can be further configured to determine that the thoracic impedance estimate meets fluid status condition criteria, detect a fluid status condition in response to the thoracic impedance estimate meeting the fluid status condition criteria, generate an output in response to detecting the fluid status condition, and store data relating to the thoracic impedance estimate in response to the generated output.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIGS. 1A and 1B are conceptual diagrams of a medical device system configured to monitor thoracic impedance according to one example.

FIGS. 2A-2C are conceptual diagrams of a patient implanted with the medical device system of FIG. 1A in a different implant configuration than the arrangement shown in FIGS. 1A-1B.

FIG. 3 is a conceptual diagram of a medical device configured to monitor thoracic impedance according to one example.

FIG. 4 is a flow chart of a method that may be performed by a medical device for monitoring thoracic impedance according to one example.

FIG. 5A is a conceptual diagram 200 of impedance measurement electrode vectors and a desired thoracic impedance pathway.

FIG. 5B is a diagram of a circuit model of thoracic impedance that may be used in computing a thoracic impedance estimate based on the impedance measurements represented in FIG. 5A according to one example.

FIG. 6 is a graph of impedances measured along a desired thoracic impedance pathway and thoracic impedance estimates corresponding to the desired impedance pathway computed using the techniques disclosed herein according to one example.

FIG. 7 is a conceptual diagram of a thoracic impedance measurement obtained using a transvenous lead (in panel A) and a conceptual diagram (in panel B) of a circuit model of thoracic impedance that may be used in computing a thoracic impedance estimate.

FIG. 8 is a graph of impedance measurements and thoracic impedance estimates determined based on a circuit model of thoracic impedance and the impedance measurements taken over time according to one example.

FIG. 9 is a graph of impedances measurements and thoracic impedance estimates determined from the impedance measurements plotted over time according to another example.

FIG. 10 is a graph of calculated impedance elements of a thoracic impedance circuit model and the thoracic impedance estimate determined as an equivalent impedance of the impedance elements according to one example.

FIG. 11 is a flow chart of a method for monitoring thoracic impedance according to one example.

FIG. 12 is a flow chart of a method for monitoring a fluid status of a patient according to another example.

FIG. 13 is a flow chart of a method that may be performed by a medical device for monitoring thoracic impedance for detecting a fluid status condition according to another example.

FIG. 14 is diagram of a method for selecting a circuit model of impedance and selecting impedance elements of the circuit model for computing the impedance through a tissue or body region.

FIG. 15 is a diagram of another example of a user interface that may be displayed by a display unit of an external device for enabling a user to select one or more of the impedance measurement electrodes, impedance circuit model and/or circuit model impedance elements used by the medical device for computing a tissue impedance.

### DETAILED DESCRIPTION

In general, this disclosure describes a medical device and techniques for monitoring impedance of body tissue or a body region. Electrical impedance of a body tissue or region may decrease with increased fluid content, e.g., edema, and increase with decreased fluid content, e.g., as edema is resolved or dehydration develops. In the examples presented herein, impedance measurements and determination of an impedance estimate from the impedance measurement for thoracic impedance monitoring are described. Thoracic impedance decreases with increasing edema, e.g., due to congestive heart failure, and increases with decreasing edema, e.g., in response to diuretics or heart failure therapies. Changes in thoracic impedance, therefore, may provide an indication of the heart failure state of a patient and enable early medical intervention, which may be to address under-diuresis, over-diuresis or optimize a heart failure therapy such as pharmaceutical therapies or CRT. While the illustrative examples presented herein refer to thoracic impedance monitoring, which may be useful in heart failure monitoring and therapy management applications, the disclosed techniques may be used in monitoring the fluid status of other body tissues or regions for detecting changes in fluid status relating to other conditions or disease, such as kidney disorders, dehydration, etc. Detection of a change in impedance of a body tissue or region may provide an early warning of a change in fluid status associated with a disease state or condition, which may trigger a therapy adjustment by a medical device or transmission of a notification or alert to the patient or a clinician or caregiver, enabling a prompt and appropriate response.

In the examples presented herein, multiple impedance measurements are obtained and used in computing an estimated impedance based on a circuit model of the impedance of the body tissue or region of interest. For example, an equivalent impedance of a three terminal wye (or “Y”) circuit model may represent the thoracic impedance. An equivalent impedance of the circuit model is an impedance presented by a combination of individual impedance elements included in the circuit model, which may include a series and/or parallel combination of the individual impedance elements. The equivalent impedance and/or individual impedance elements of the circuit model of the impedance of the body tissue or region may not correspond well to available impedance measurement electrode vectors in some clinical applications. For example, electrode placement for optimizing sensing of cardiac electrical signals, delivery of cardiac electrical stimulation therapies, anatomical limitations or other considerations may result in implanted locations of electrodes that result in impedance measurement electrode vector pathways that are not ideal for measuring and monitoring thoracic impedance e.g., for monitoring a fluid condition.

Techniques disclosed herein provide methods for determining a thoracic impedance estimate according to a circuit model of thoracic impedance using multiple impedance measurements where at least one measurement corresponds to a series and/or parallel combination of the individual impedance elements of the circuit model. Each of the impedance measurements do not necessarily correspond to an individual impedance element of the circuit model or to the equivalent impedance of the circuit model. However, the available impedance measurements may be used to compute a thoracic impedance estimate as an equivalent impedance and/or an impedance of one or more individual impedance elements of the circuit model, particularly when electrode implant locations may limit the ability to obtain reliable, precise or accurate thoracic impedance measurements.

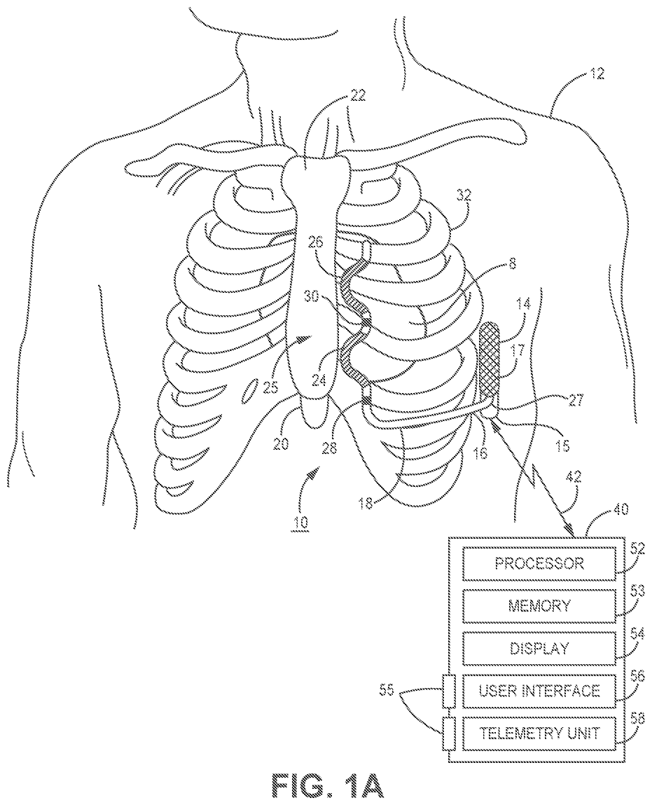

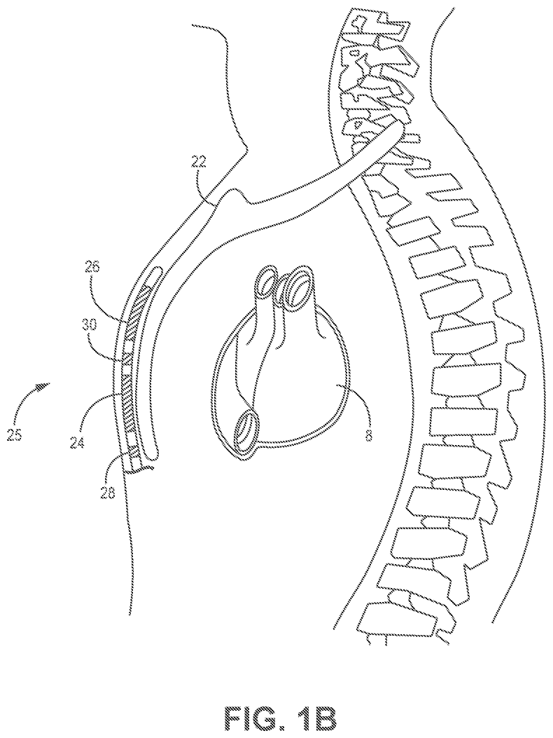

FIGS. 1A and 1B are conceptual diagrams of a medical device system 10 configured to monitor thoracic impedance according to one example. The system 10 includes an implantable cardioverter defibrillator (ICD) 14 connected to an extra-cardiac electrical stimulation and sensing lead 16. FIG. 1A is a front view of ICD 14 implanted within patient 12. FIG. 1B is a side view of ICD 14 implanted within patient 12. In this example, ICD 14 is configured to sense cardiac electrical signals and deliver cardiac electrical stimulation therapies, which may include cardiac pacing and/or cardioversion/defibrillation shock therapies (CV/DF).

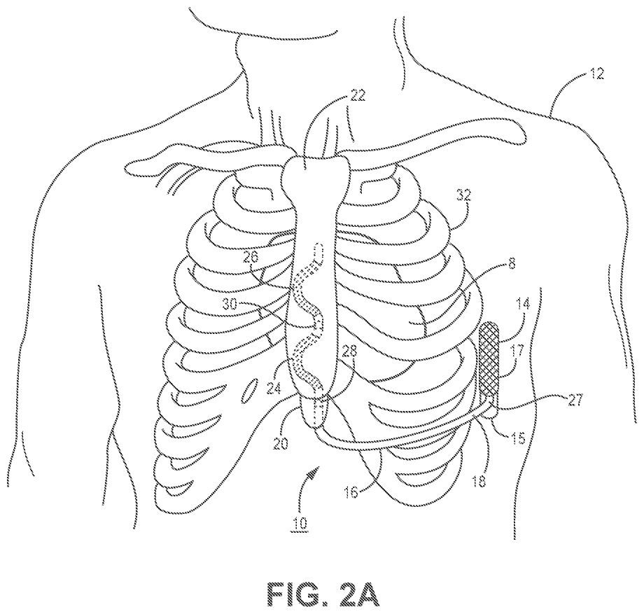

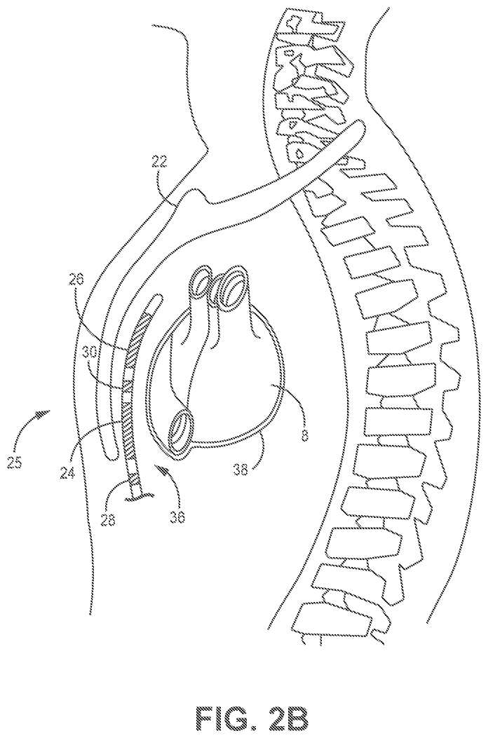

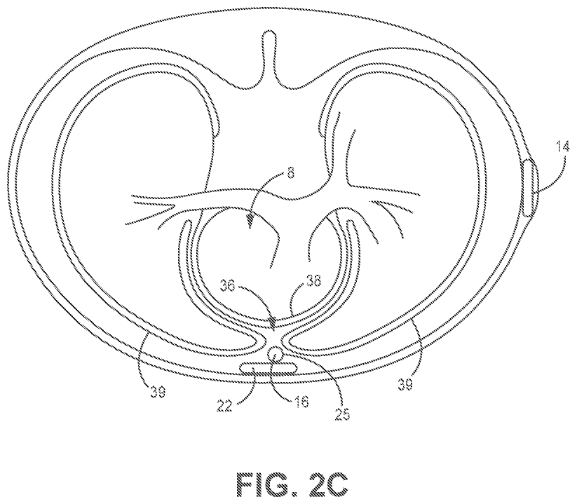

In this example, lead 16 is a non-transvenous, extra-cardiovascular electrical stimulation and sensing lead 16 that may be used for sensing impedance signals to obtain impedance measurements using multiple impedance measurement electrode vectors. The impedance measurements may be used for determining a thoracic impedance estimate according to an electrical circuit model of thoracic impedance. ICD 14 may be coupled to a transvenous or non-transvenous lead carrying electrodes for obtaining impedance measurements, sensing cardiac electrical signals, and delivering electrical stimulation therapy in various examples. For instance, lead 16 may be an “extra-cardiovascular” lead, referring to a lead that is positioned outside the blood vessels, heart, and pericardium surrounding the heart of a patient. Implantable electrodes carried by extra-cardiovascular leads may be positioned extra-thoracically (outside the ribcage and sternum) or intra-thoracically (beneath the ribcage or sternum, sometimes referred to as a sub-sternal position) but not necessarily in intimate contact with myocardial tissue, e.g., outside the pericardium. Examples of extra-thoracic and intra-thoracic positions of lead 16 are described in conjunction with FIGS. 1A-2C. An extra-cardiovascular lead may also be referred to as a “non-transvenous” lead. In these examples, multiple extra-cardiac electrode vectors are available for obtaining impedance measurements for determining a thoracic impedance estimate.

In other examples, ICD 14 may be coupled to a transvenous lead that positions electrodes within a blood vessel, which may remain outside the heart in an extra-cardiac location. For instance, a transvenous medical lead coupled to ICD 14 may be advanced along a venous pathway to position electrodes in an extra-cardiac location within the internal thoracic vein (ITV), an intercostal vein, the superior epigastric vein, or the azygos, hemiazygos, or accessory hemiazygos veins, as examples. In still other examples, ICD 14 may be coupled to a transvenous lead advanced to position electrodes within the heart, e.g., within an atrial and/or ventricular heart chamber, or a lead positioned on the epicardium or within the pericardium of the heart 8. The transvenous lead may be coupled to ICD 14 in combination with a second non-transvenous lead such that multiple impedance measurement electrode vectors for obtaining impedance measurements are available.

ICD 14 includes a housing 15 that forms a hermetic seal that protects internal components of ICD 14. The housing 15 of ICD 14 may be formed of a conductive material, such as titanium or titanium alloy. The housing 15 may function as an electrode (sometimes referred to as a “can” electrode). The housing 15 may be used in combination with electrodes carried by lead 16 for obtaining impedance measurements from multiple impedance measurement electrode vectors for thoracic impedance monitoring. In some examples, housing 15 may be used as an active can electrode for use in delivering CV/DF shocks or other high voltage pulses delivered using a high voltage therapy circuit. In other examples, housing 15 may be available for use in delivering unipolar, low voltage cardiac pacing pulses and/or for sensing cardiac electrical signals in combination with electrodes carried by lead 16. In other instances, the housing 15 of ICD 14 may include a plurality of electrodes on an outer portion of the housing. The outer portion(s) of the housing 15 functioning as an electrode(s) may be coated with a material, such as titanium nitride, e.g., for reducing post-stimulation polarization artifact.

ICD 14 includes a connector assembly 17 (also referred to as a connector block or header) that includes electrical feedthroughs crossing housing 15 to provide electrical connections between conductors extending within the lead body 18 of lead 16 and electronic components included within the housing 15 of ICD 14. As will be described in further detail herein, housing 15 may house one or more processors, memories, transceivers, cardiac electrical signal sensing circuitry, impedance measurement circuitry, therapy delivery circuitry, power sources and other components for performing various functions of ICD 14, which may include obtaining impedance measurements, determining thoracic impedance estimates, sensing cardiac electrical signals, detecting a heart rhythm, and controlling and delivering electrical stimulation pulses to treat an abnormal heart rhythm and/or heart failure.

Lead 16 is shown in this example as an extra-cardiovascular lead implanted outside the ribcage and sternum. Lead 16 includes an elongated lead body 18 having a proximal end 27 that includes a lead connector (not shown) configured to be connected to ICD connector assembly 17 and a distal portion 25 that includes one or more electrodes. In the example illustrated in FIGS. 1A and 1B, the distal portion 25 of lead body 18 includes defibrillation electrodes 24 and 26 and pace/sense electrodes 28 and 30. In some cases, defibrillation electrodes 24 and 26 may together form a defibrillation electrode in that they may be configured to be activated concurrently. Alternatively, defibrillation electrodes 24 and 26 may form separate defibrillation electrodes in which case each of the electrodes 24 and 26 may be activated independently. In the illustrative examples presented herein, electrodes 24 and 26 may be selected individually or in combination for obtaining impedance measurements from multiple electrode vectors. For instance, an impedance measurement may be obtained between electrode 24 and housing 15, between electrode 26 and housing 15, between electrodes 24 and 26, between electrode 24 and a combination of electrode 26 and housing 15, between electrode 26 and a combination of electrode 24 and housing 15, or between a combination of electrodes 24 and 26 and housing 15 as examples. A thoracic impedance estimate that is correlated to thoracic fluid status may be determined from at least three impedance measurements according to a circuit model of thoracic impedance as further described below.

Electrodes 24 and 26 (and in some examples housing 15) are referred to herein as defibrillation electrodes because they may be utilized, individually or collectively, for delivering high voltage stimulation therapy (e.g., cardioversion or defibrillation shocks). Electrodes 24 and 26 may be elongated coil electrodes and generally have a relatively high surface area for delivering high voltage electrical stimulation pulses compared to pacing and sensing electrodes 28 and 30. However, electrodes 24 and 26 and housing 15 may also be utilized to provide pacing functionality, sensing functionality and/or impedance monitoring in addition to or instead of high voltage stimulation therapy. In this sense, the use of the term “defibrillation electrode” herein should not be considered as limiting the electrodes 24 and 26 for use in only high voltage cardioversion/defibrillation shock therapy applications. For example, in addition to obtaining impedance measurements, either of electrodes 24 and 26 may be used as a sensing electrode in a sensing electrode vector for sensing cardiac electrical signals and determining a need for an electrical stimulation therapy. Electrodes 24 and/or 30 may be used in delivering a drive signal, sensing a resulting impedance signal or both.

Electrodes 28 and 30 are relatively smaller surface area electrodes which are available for use in sensing electrode vectors for sensing cardiac electrical signals and may be used for delivering relatively low voltage cardiac pacing pulses in some configurations. Electrodes 28 and 30 are referred to herein as “pace/sense electrodes” because they are generally configured for use in low voltage applications, e.g., used as either a cathode or anode for delivery of pacing pulses and/or sensing of cardiac electrical signals, as opposed to delivering high voltage CV/DF shocks. In some instances, electrodes 28 and 30 may be used in impedance measurement electrode vectors, which may include delivering a drive signal and/or recording the resultant impedance signal. Electrodes 28 and 30 may provide only pacing functionality, only sensing functionality, only impedance measurement functionality or any combination thereof.

Various impedance measurement electrode vectors may be selected from the electrodes 24, 26, 28 and 30 and housing 15 for obtaining multiple impedance measurements that are used to determine a thoracic impedance estimate using the techniques disclosed herein. In addition to the examples given above, impedance measurement electrode vectors may include the vector between electrodes 28 and 30, electrode 28 and housing 15, electrode 30 and housing 15, or between one of electrodes 28 or 30 and either or the combination of both defibrillation electrodes 24 and/or 26, as examples.

In addition to obtaining impedance measurements, ICD 14 may sense cardiac electrical signals corresponding to electrical activity of heart 8 via one or more sensing electrode vectors that include combinations of electrodes 24, 26, 28 and/or 30. In some examples, housing 15 of ICD 14 is used in combination with one or more of electrodes 24, 26, 28 and/or 30 in a sensing electrode vector. In the example illustrated in FIGS. 1A and 1B, electrode 28 is located proximal to defibrillation electrode 24, and electrode 30 is located between defibrillation electrodes 24 and 26. One, two or more pace/sense electrodes may be carried by lead body 18. For instance, a third pace/sense electrode may be located distal to defibrillation electrode 26 in some examples. Electrodes 28 and 30 are illustrated as ring electrodes; however, electrodes 28 and 30 may comprise any of a number of different types of electrodes, including ring electrodes, short coil electrodes, hemispherical electrodes, directional electrodes, segmented electrodes, or the like. Electrodes 28 and 30 may be positioned at other locations along lead body 18 and are not limited to the positions shown. In other examples, lead 16 may include fewer or more pace/sense electrodes and/or defibrillation electrodes than the example shown here.

In some examples, the electrodes coupled to ICD 14 or another medical device configured to obtain impedance measurements for determining an impedance estimate through a body tissue or region include at least three electrodes (one of which may be the medical device housing) arranged in an approximately triangular configuration. As described below, each electrode may correspond to a terminal of a circuit model of impedance through the tissue or body region. For example, three electrodes may correspond to a three terminal circuit model of thoracic impedance, which may correspond to a wye circuit in some examples. In this way, an impedance measurement from each one of multiple impedance measurement electrode vectors may be used to compute an impedance of the circuit model as an estimate of the impedance through the corresponding body tissue or body cavity. In the case of ICD 14 coupled to lead 16, two electrodes carried by lead 16 and the housing 15 may correspond to three terminals of a circuit model of thoracic impedance. Using three impedance measurements obtained using three different measurement vectors selected from the two electrodes on lead 16 and housing 15, the equivalent impedance of a combination of the individual impedance elements of the circuit model may be computed as an estimate of thoracic impedance as further described below.

Lead 16 may extend subcutaneously or submuscularly over the ribcage 32 medially from the connector assembly 27 of ICD 14 toward a center of the torso of patient 12, e.g., toward xiphoid process 20 of patient 12. At a location near xiphoid process 20, lead 16 may bend or turn to extend superiorly, subcutaneously or submuscularly, over the ribcage and/or sternum, substantially parallel to sternum 22. Although illustrated in FIG. 1A as being offset laterally from and extending substantially parallel to sternum 22, the distal portion 25 of lead 16 may be implanted at other locations, such as over sternum 22, offset to the right or left of sternum 22, angled laterally from sternum 22 toward the left or the right, or the like. Alternatively, lead 16 may be placed along other subcutaneous or submuscular paths. The path of non-transvenous, extra-cardiovascular lead 16 may depend on the location of ICD 14, the arrangement and position of electrodes carried by the lead body 18, and/or other factors. The techniques disclosed herein are not limited to a particular path of lead 16 or final locations of electrodes 24, 26, 28 and 30. The techniques for determining a thoracic impedance estimate from a combination of multiple impedance measurements, e.g., at least three impedance measurements obtained from each one of three respective impedance measurement electrode vectors, may be tailored to the relative positions of the electrodes such that a circuit model of impedance having terminals corresponding to each of the electrodes included in the impedance measurement electrode vectors is representative of a desired impedance pathway extending through at least a portion of the thoracic cavity (or other body tissue or region of interest).

Electrical conductors (not illustrated) extend through one or more lumens of the elongated lead body 18 of lead 16 from the lead connector at the proximal lead end 27 to electrodes 24, 26, 28, and 30 located along the distal portion 25 of the lead body 18. The elongated electrical conductors contained within the lead body 18, which may be separate respective insulated conductors within the lead body 18, are each electrically coupled with respective defibrillation electrodes 24 and 26 and pace/sense electrodes 28 and 30. The respective conductors electrically couple the electrodes 24, 26, 28, and 30 to circuitry, such as a therapy delivery circuit and/or a cardiac electrical signal sensing circuit, of ICD 14 via connections in the connector assembly 17, including associated electrical feedthroughs crossing housing 15. The electrical conductors transmit therapy from a therapy delivery circuit within ICD 14 to one or more of defibrillation electrodes 24 and 26 and/or pace/sense electrodes 28 and 30 and transmit cardiac electrical signals sensed from the patient's heart 8 from one or more of defibrillation electrodes 24 and 26 and/or pace/sense electrodes 28 and 30 to the sensing circuit within ICD 14.

For the purposes of obtaining impedance measurements, the therapy delivery circuit (or a dedicated impedance drive signal circuit) may be coupled to a selected electrode vector for delivering a drive signal, which may be a voltage-controlled or current-controlled signal. In illustrative examples presented herein, the drive signal is a current-controlled signal, e.g., a 0.1 ms current pulse having a current amplitude in the range of 0.02 to 0.1 milliamperes, as examples. The resulting voltage across the measurement electrode vector may be determined as the impedance measurement or converted to an actual impedance based on the injected current and the resulting voltage. In other examples, the drive signal is a voltage-controlled signal and the resulting impedance signal may be sensed as the voltage change of a holding capacitor used to generate the drive signal as it is discharged across the selected impedance measurement electrode vector. The voltage change may be obtained as an impedance measurement since the change in voltage over a given pulse width is dependent on the current flow, which is dependent on the tissue impedance between the electrodes included in the impedance measurement electrode vector. Thus the voltage at the end of a voltage-controlled electrical pulse (or the change in voltage from the beginning to the end of the voltage-controlled electrical pulse) may be obtained as the impedance measurement for a given impedance electrode vector. In other examples, separate drive signal electrode vectors and recording electrode vectors may be selected for sensing impedance signals for monitoring thoracic impedance.

The lead body 18 of lead 16 may be formed from a non-conductive material, including silicone, polyurethane, fluoropolymers, mixtures thereof, and/or other appropriate materials, and shaped to form one or more lumens within which the one or more conductors extend. Lead body 18 may be tubular or cylindrical in shape. In other examples, the distal portion 25 (or all of) the elongated lead body 18 may have a flat, ribbon or paddle shape. Lead body 18 may be formed having a preformed distal portion 25 that is generally straight, curving, bending, serpentine, undulating or zig-zagging.

In the example shown, lead body 18 includes a curving distal portion 25 having two “C” shaped curves, which together may resemble the Greek letter epsilon, “c.” Defibrillation electrodes 24 and 26 are each carried by one of the two respective C-shaped portions of the lead body distal portion 25. The two C-shaped curves are seen to extend or curve in the same direction away from a central axis of lead body 18, along which pace/sense electrodes 28 and 30 are positioned. Pace/sense electrodes 28 and 30 may, in some instances, be approximately aligned with the central axis of the proximal portion of lead body 18 when laid straight such that mid-points of defibrillation electrodes 24 and 26 are laterally offset from pace/sense electrodes 28 and 30.

Other examples of extra-cardiovascular leads including one or more defibrillation electrodes and one or more pacing and sensing electrodes may include a curving, serpentine, undulating or zig-zagging distal portion of the lead body 18. The techniques disclosed herein are not limited to any particular lead body design, however. In other examples, lead body 18 is a flexible elongated lead body without any pre-formed shape, bends or curves.

ICD 14 analyzes the cardiac electrical signals received from one or more sensing electrode vectors to monitor for abnormal rhythms, such as bradycardia, ventricular tachycardia (VT) or ventricular fibrillation (VF). ICD 14 may analyze the rate of sensed cardiac event signals (e.g., R-waves attendant to ventricular depolarizations) and the morphology of the cardiac electrical signals to monitor for tachyarrhythmia in accordance with any of a number of tachyarrhythmia detection techniques. ICD 14 generates and delivers electrical stimulation therapy in response to detecting a tachyarrhythmia (e.g., VT or VF) using a therapy delivery electrode vector which may be selected from any of the available electrodes 24, 26, 28 30 and/or housing 15. ICD 14 may deliver anti-tachycardia pacing (ATP) in response to VT detection and in some cases may deliver ATP prior to a CV/DF shock or during high voltage capacitor charging in an attempt to avert the need for delivering a CV/DF shock. If ATP does not successfully terminate VT or when VF is detected, ICD 14 may deliver one or more CV/DF shocks via one or both of defibrillation electrodes 24 and 26 and/or housing 15. ICD 14 may deliver the CV/DF shocks using electrodes 24 and 26 individually or together as a cathode (or anode) and with the housing 15 as an anode (or cathode). ICD 14 may generate and deliver other types of electrical stimulation pulses such as post-shock pacing pulses, asystole pacing pulses, CRT, or bradycardia pacing pulses using a pacing electrode vector that includes one or more of the electrodes 24, 26, 28, and 30 and the housing 15 of ICD 14.

ICD 14 is shown implanted subcutaneously on the left side of patient 12 along the ribcage 32. ICD 14 may, in some instances, be implanted between the left posterior axillary line and the left anterior axillary line of patient 12. ICD 14 may, however, be implanted at other subcutaneous or submuscular locations in patient 12. For example, ICD 14 may be implanted in a subcutaneous pocket in the pectoral region. In this case, lead 16 may extend subcutaneously or submuscularly from ICD 14 toward the manubrium of sternum 22 and bend or turn and extend inferiorly from the manubrium to the desired location subcutaneously or submuscularly. In yet another example, ICD 14 may be placed abdominally. Lead 16 may be implanted in other extra-cardiovascular locations as well. For instance, as described with respect to FIGS. 2A-2C, the distal portion 25 of lead 16 may be implanted underneath the sternum/ribcage in the substernal space.

Referring to FIG. 1A, an external device 40 is shown in telemetric communication with ICD 14 by a communication link 42. External device 40 may include a processor 52, memory 53, display unit 54, user interface 56 and telemetry unit 58. Processor 52 controls external device operations and processes data and signals received from ICD 14. Processor 52 executes instructions stored in memory 53. Processor 52 may include any one or more of a microprocessor, a controller, a digital signal processor (DSP), an application specific integrated circuit (ASIC), a field-programmable gate array (FPGA), or equivalent discrete or analog logic circuitry. In some examples, processor 52 may include multiple components, such as any combination of one or more microprocessors, one or more controllers, one or more DSPs, one or more ASICs, or one or more FPGAs, as well as other discrete or integrated logic circuitry. The functions attributed to processor 52 herein may be embodied as software, firmware, hardware or any combination thereof.

Memory 53 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Memory 53 may be configured to store control parameters and associated programmable settings used in programming ICD 14 and used by ICD 14 in controlling cardiac signal sensing, impedance monitoring and therapy delivery functions. Memory 53 may store data received by or determined by processor 52 for use in generating an output representative of thoracic impedance changes according to the techniques disclosed herein.

Display unit 54, which may include a graphical user interface, displays data and other information to a user for reviewing ICD operation and programmed parameters as well as cardiac electrical signals and/or impedance related data retrieved from ICD 14. As described below, a processor of ICD 14 or external device processor 52 may determine a thoracic impedance estimate from impedance measurements obtained by ICD 14. The thoracic impedance estimate may be compared to criteria for detecting a fluid status condition, e.g., edema, dehydration or over-diuresis. External device processor 52 may generate data output for display by display unit 54, which may include graphical representations of the thoracic impedance estimates or a fluid status index derived therefrom over time and/or relative to a predetermined threshold or range corresponding to normal thoracic fluid status. In this way, information may be presented to a user related to changes in thoracic impedance over time, which may include detection of a fluid status condition such as edema or over-diuresis. External device processor 52 may receive impedance measurements, thoracic impedance estimates, fluid status condition detection alerts or notifications or any combination thereof for use in generating an output, e.g., for display on display unit 54, presenting information relating to the patient's fluid status.

User interface 56 may include a mouse, touch screen, keypad or the like to enable a user to interact with external device 40 to initiate a telemetry session with ICD 14 for retrieving data from and/or transmitting data to ICD 14, including programmable parameters for controlling cardiac event sensing and therapy delivery and for thoracic impedance monitoring. A clinician may use user interface 56 to send and receive commands to ICD 14 via external device 40. Typically, user interface 56 includes one or more input devices and one or more output devices, including display unit 54. The input devices of user interface 56 may include a communication device such as a network interface, keyboard, pointing device, voice responsive system, video camera, biometric detection/response system, button, sensor, mobile device, control pad, microphone, presence-sensitive screen, touch-sensitive screen (which may be included in display unit 54), network, or any other type of device for detecting input from a human or machine.

The one or more output devices of user interface 56 may include a communication unit such as a network interface, display, sound card, video graphics adapter card, speaker, presence-sensitive screen, one or more USB interfaces, video and/or audio output interfaces, or any other type of device capable of generating tactile, audio, video, or other output. Display unit 54 may function as an input and/or output device using technologies including liquid crystal displays (LCD), quantum dot display, dot matrix displays, light emitting diode (LED) displays, organic light-emitting diode (OLED) displays, cathode ray tube displays, e-ink, or monochrome, color, or any other type of display capable of generating tactile, audio, and/or visual output. In other examples, user interface 56 may produce an output to a user in another fashion, such as via a sound card, video graphics adapter card, speaker, presence-sensitive screen, touch-sensitive screen, one or more USB interfaces, video and/or audio output interfaces, or any other type of device capable of generating tactile, audio, video, or other output. In some examples, display unit 54 is a presence-sensitive display that may serve as a user interface device that operates both as one or more input devices and one or more output devices.

Telemetry unit 58 includes a transceiver and antenna configured for bidirectional communication with a telemetry circuit included in ICD 14 and is configured to operate in conjunction with processor 52 for sending and receiving data relating to ICD functions via communication link 42. Communication link 42 may be established between ICD 14 and external device 40 using a radio frequency (RF) link such as BLUETOOTH®, Wi-Fi, or Medical Implant Communication Service (MICS) or other RF or communication frequency bandwidth or communication protocols. Data stored or acquired by ICD 14, including cardiac electrical signals and impedance measurements or associated data derived therefrom, results of device diagnostics, and histories of detected rhythm episodes and delivered therapies, may be retrieved from ICD 14 by external device 40 following an interrogation command. For instance, external device 40 may retrieve episodes of sensed cardiac electrical signals and/or thoracic impedance estimates or related data from ICD 14. Based on the thoracic impedance data, processor 52 may generate an alert or notification output by user interface 56 indicating that a condition relating to the patient's thoracic impedance may warrant medical attention.

External device 40 may be used to program sensing control parameters, impedance monitoring control parameters, cardiac rhythm detection parameters and therapy delivery control parameters used by ICD 14. External device 50 may include external ports 55 for electrical connection to surface ECG leads and electrodes (not shown in FIG. 1A) that may be coupled to a patient implanted with ICD 14. Processor 52 may receive ECG signals for display by display unit 54 for observation by a user. External device 40 may be embodied as a programmer used in a hospital, clinic or physician's office to retrieve data from ICD 14 and to program operating parameters and algorithms in ICD 14 for controlling ICD functions. External device 40 may alternatively be embodied as a home monitor or handheld device, which may be a tablet, cell phone or other personal device. While external device 40 is shown only in FIG. 1A in communication with ICD 14, it is to be understood that portions of the techniques disclosed herein may be performed by an external device, such as device 40, configured to communicate with an implantable or another external medical device configured to sense and transmit signals to external device 40. Aspects of external device 40 may generally correspond to the external programming/monitoring unit disclosed in U.S. Pat. No. 5,507,782 (Kieval, et al.), hereby incorporated herein by reference in its entirety. An example programmer that may be configured to perform the techniques disclosed herein is the CARELINK® Programmer, commercially available from Medtronic, Inc., Minneapolis, Minn., USA.

FIGS. 2A-2C are conceptual diagrams of patient 12 implanted with ICD system 10 in a different implant configuration than the arrangement shown in FIGS. 1A-1B. FIG. 2A is a front view of patient 12 implanted with ICD system 10. FIG. 2B is a side view of patient 12 implanted with ICD system 10. FIG. 2C is a transverse view of patient 12 implanted with ICD system 10. In this arrangement, lead 16 of system 10 is implanted at least partially underneath sternum 22 of patient 12. Lead 16 extends subcutaneously or submuscularly from ICD 14 toward xiphoid process 20 and at a location near xiphoid process 20 bends or turns and extends superiorly within anterior mediastinum 36 in a substernal position.

Anterior mediastinum 36 may be viewed as being bounded laterally by pleurae 39, posteriorly by pericardium 38, and anteriorly by sternum 22 (see FIG. 2C). The distal portion 25 of lead 16 may extend along the posterior side of sternum 22 substantially within the loose connective tissue and/or substernal musculature of anterior mediastinum 36. A lead implanted such that the distal portion 25 is substantially within anterior mediastinum 36, or within a pleural cavity or more generally within the thoracic cavity, may be referred to as a “substernal lead.”

In the example illustrated in FIGS. 2A-2C, lead 16 is located substantially centered under sternum 22. In other instances, however, lead 16 may be implanted such that it is offset laterally from the center of sternum 22. In some instances, lead 16 may extend laterally such that distal portion 25 of lead 16 is underneath/below the ribcage 32 in addition to or instead of sternum 22. In other examples, the distal portion 25 of lead 16 may be implanted in other extra-cardiovascular, intra-thoracic locations, including the pleural cavity or around the perimeter of and adjacent or within the pericardium 38 of heart 8. In the examples described below in conjunction with FIGS. 1A-2C, electrodes for sensing cardiac electrical signals and obtaining impedance measurements are carried by a lead that may be advanced to a supra-diaphragmatic position, which may be within the thoracic cavity or outside the thorax in various examples.

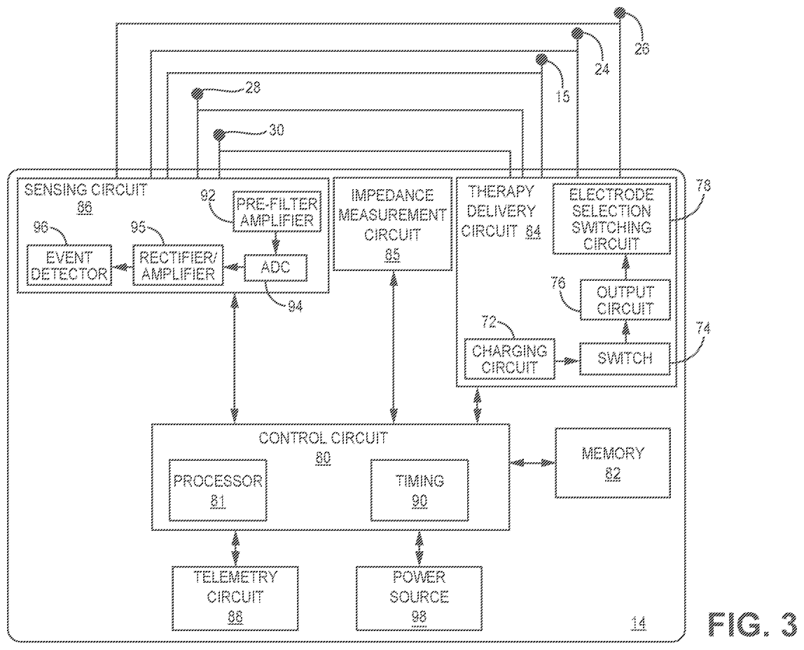

FIG. 3 is a conceptual diagram of a medical device configured to monitor thoracic impedance according to one example. FIG. 3 is described in conjunction with the ICD 14 of FIGS. 1A-2C, including therapy delivery capabilities. It is to be understood, however, the circuitry and functionality attributed to circuitry described in conjunction with FIG. 3 may be included, in whole or in part, in a cardiac monitoring device that does not have therapy delivery capabilities in some examples. The ICD housing 15 is shown schematically as an electrode in FIG. 3 since the housing of the medical device may be used as an electrode, e.g., in an electrode vector for monitoring thoracic impedance, sensing cardiac electrical signals and/or for therapy delivery. The electronic circuitry enclosed within housing 15 includes software, firmware and/or hardware that cooperatively monitor cardiac signals, determine when an electrical stimulation therapy is necessary, and deliver therapy as needed according to programmed therapy delivery algorithms and control parameters. According to the techniques disclosed herein, the software, firmware and/or hardware is configured to sense multiple impedance signals and determine a thoracic impedance estimate, e.g., for providing heart failure monitoring.

ICD 14 as shown in FIG. 3 includes a control circuit 80, memory 82, therapy delivery circuit 84, impedance measurement circuit 85, cardiac electrical signal sensing circuit 86, and telemetry circuit 88. A power source 98 provides power to the circuitry of ICD 14, including each of the components 80, 82, 84, 85, 86, and 88 as needed. Power source 98 may include one or more energy storage devices, such as one or more rechargeable or non-rechargeable batteries. The connections between power source 98 and each of the other components 80, 82, 84, 85, 86 and 88 are to be understood from the general block diagram of FIG. 3 but are not shown for the sake of clarity. For example, power source 98 may be coupled to one or more charging circuits included in therapy delivery circuit 84 for charging holding capacitors or other charge storage devices included in therapy delivery circuit 84 that are discharged at appropriate times under the control of control circuit 80 for producing electrical pulses according to a therapy protocol. In other examples, power source 98 may serve as a voltage or current source to therapy delivery circuit 84 and/or impedance measurement circuit 85 without requiring a charge storage device. Power source 98 is also coupled to components of cardiac electrical signal sensing circuit 86, such as sense amplifiers, analog-to-digital converters, switching circuitry, etc. as needed. Power source 98 provides power for delivering a drive signal to selected impedance measurement electrode vectors for obtaining impedance measurements.

The circuits shown in FIG. 3 represent functionality included in ICD 14 or another medical device operating according to the techniques disclosed herein and may include any discrete and/or integrated electronic circuit components that implement analog and/or digital circuits capable of producing the functions attributed to ICD 14 herein. Functionality associated with one or more circuits may be performed by separate hardware, firmware or software components, or integrated within common hardware, firmware or software components.

For example, impedance measurements may be performed by a dedicated impedance measurement circuit 85, which may include circuitry for generating a drive signal powered by power source 98 and recording a resulting impedance signal from an impedance measurement electrode vector. In other examples, impedance measurements may be performed by therapy delivery circuit 84 and control circuit 80 without requiring a dedicated impedance measurement circuit 85 or cooperatively by therapy delivery circuit 84 and impedance measurement circuit 85 in various device configurations. For example, impedance measurement circuit 85 may generate a current drive signal and measure the resulting voltage across an impedance measurement electrode vector. In another example, therapy delivery circuit 84 may generate a voltage pulse that is delivered across a selected impedance measurement electrode vector and impedance measurement circuit 85 (or control circuit 80) may receive a voltage signal from therapy delivery circuit 84 corresponding to the voltage remaining on a holding capacitor at the end of the voltage pulse. Accordingly, determination of a thoracic impedance estimate may be performed cooperatively by therapy delivery circuit 84, impedance measurement circuit 84 and/or control circuit 80 and may include operations implemented in a processor or other signal processing circuitry included in control circuit 80 executing instructions stored in memory 82.

The various circuits of ICD 14 may include an application specific integrated circuit (ASIC), an electronic circuit, a processor (shared, dedicated, or group) and memory that execute one or more software or firmware programs, a combinational logic circuit, state machine, or other suitable components or combinations of components that provide the described functionality. The particular form of software, hardware and/or firmware employed to implement the functionality disclosed herein will be determined primarily by the particular system architecture employed in the medical device and by the particular sensing and therapy delivery methodologies employed by the medical device. Providing software, hardware, and/or firmware to accomplish the described functionality in the context of any modern medical device, given the disclosure herein, is within the abilities of one of skill in the art.

Memory 82 may include any volatile, non-volatile, magnetic, or electrical non-transitory computer readable storage media, such as random access memory (RAM), read-only memory (ROM), non-volatile RAM (NVRAM), electrically-erasable programmable ROM (EEPROM), flash memory, or any other memory device. Furthermore, memory 82 may include non-transitory computer readable media storing instructions that, when executed by one or more processing circuits, cause control circuit 80 and/or other ICD components to perform various functions attributed to ICD 14 or those ICD components. The non-transitory computer-readable media storing the instructions may include any of the media listed above.

Control circuit 80 communicates, e.g., via a data bus, with therapy delivery circuit 84, impedance measurement circuit 85, and sensing circuit 86 for monitoring thoracic impedance, sensing cardiac signals, detecting cardiac rhythms, and controlling delivery of cardiac electrical stimulation therapies. Therapy delivery circuit 84, impedance measurement circuit 85 and sensing circuit 86 are electrically coupled to electrodes 24, 26, 28, 30 and the housing 15, which may function as a common or ground electrode or as an active can electrode for delivering CV/DF shock pulses or cardiac pacing pulses. As described above electrodes 24, 26, 28 and 30 shown in FIG. 3 may be carried by a non-transvenous lead advanced to position electrodes in an extra-cardiac location (as shown in FIGS. 1A-2C) or by a transvenous lead for positioning electrodes within a blood vessel. Furthermore, electrodes coupled to ICD 14 may include multiple housing-based electrodes not carried by a lead in some examples.

Impedance measurement circuit 85 may obtain impedance measurements from multiple impedance measurement electrode vectors selected from the available electrodes 24, 26, 28, 30 and housing 15. In one example, impedance measurement circuit 85 may include drive signal circuitry and impedance measurement circuitry for delivering a voltage or current controlled drive signal and record a resulting impedance signal (e.g., the resulting current or voltage signal) from a selected impedance measurement electrode vector. The drive signal may be delivered across the impedance measurement electrode vector or a different drive signal electrode vector. In other examples, impedance measurement circuit 85 may obtain an impedance measurement by controlling therapy delivery circuit 84 to deliver an impedance measurement drive signal, e.g., a voltage or current pulse, and impedance measurement circuit 85 may receive a resulting impedance signal sensed from an impedance measurement electrode vector or multiple impedance measurement electrode vectors simultaneously.

Circuitry for performing impedance measurements and delivering therapeutic electrical stimulation pulses may be shared between impedance measurement circuit 85 and therapy delivery circuit 84, or therapy delivery circuit 84 may perform the functionality represented by impedance measurement circuit 85, without requiring separate dedicated impedance measurement circuitry. Therapy delivery circuit 84 may include a charging circuit 72, switch 74, output circuit 76 and electrode selection switching circuit 78. Charging circuit 72 may include one or more holding capacitors that are charged by power source 98 to a voltage amplitude according to a programmed pulse amplitude, which may be a therapeutic stimulation pulse amplitude or a voltage-controlled or current-controlled impedance drive signal pulse amplitude. The pulse amplitude of an impedance drive signal may be set to a “subthreshold amplitude” that is less than the capture threshold required to depolarize or “capture” the cardiac tissue, e.g., causing depolarization of the myocardium. In this way, impedance measurements may be obtained without causing cardiac capture. The holding capacitor charged to a predetermined voltage may be coupled to output circuit 76 via switch 74 for a programmed pulse width for delivering a therapeutic stimulation pulse or for delivering a subthreshold impedance measurement drive signal.

The output circuit 76 of therapy delivery circuit 84 may include a capacitor that is selectively coupled to electrodes 24, 26, 28, 30 and/or housing 15 via electrode selection switching circuit 78 according to control signals received from control circuit 80. The holding capacitor(s) of charging circuit 72 are discharged through the output circuit 76 and through the electrode vector coupled to output circuit 76 via electrode selection switching circuit 78. Electrode selection switching circuit 78 may include a switch array, switch matrix, multiplexer, or any other type of switching device(s) suitable to selectively couple selected electrodes to the output circuit 76. In this way, electrode selection switching circuit 78 may select an impedance measurement electrode vector for coupling to output circuit 76 when a drive signal pulse is being delivered for obtaining an impedance measurement. At least two measurement electrode vectors, or at least three impedance measurement electrode vectors in some examples, may be coupled to output circuit 76, e.g., during different impedance measurement time slots, to obtain multiple impedance measurements used for computing a thoracic impedance estimate. During therapy delivery, electrode selection switching circuit 78 may selectively couple a therapy delivery electrode vector selected from electrodes 24, 26, 28, 30 and housing 15 for delivering cardiac pacing pulses, CV/DF shock pulses or other therapeutic pacing pulses. In some instances, other electrical stimulation pulses may be generated and delivered by therapy delivery circuit 84, e.g., for inducing a tachyarrhythmia during CV/DF testing of ICD 14.

In some examples, when a subthreshold voltage or current pulse is delivered across a selected impedance measurement vector, the holding capacitor(s) charged to a subthreshold voltage are discharged over a drive signal pulse width. Impedance measurement circuit 85 (or control circuit 80) may receive a voltage signal on a voltage control line at the end of the drive signal pulse width. The change in voltage on the holding capacitor(s) during delivery of the drive signal pulse corresponds to the impedance of the selected impedance measurement electrode vector. The change in voltage or the ending voltage may be obtained by impedance measurement circuit 85 as an impedance measurement or may be converted to an absolute impedance measurement in ohms by impedance measurement circuit 85.

In other examples, a dedicated impedance measurement circuit 85 may be provided that is separate from or shares some components with therapy delivery circuit 84 used for therapy delivery. The impedance measurement circuit 85 may deliver a known drive signal (current or voltage) applied to a selected impedance measurement electrode vector and the resulting voltage or current signal may be sensed by an impedance measurement circuit and passed to control circuit 80. For example, a voltage signal may be sensed as the “impedance” signal or converted to an impedance value or signal using the known drive current and the resulting voltage signal by control circuit 80. In some examples, the drive signal may be applied to an impedance measurement electrode vector that is also used as a recording electrode vector for measuring impedance. In other examples, the drive signal may be applied to a drive electrode pair and the resulting impedance signal may be sensed from a recording electrode pair or multiple recording electrode pairs simultaneously. The impedance measurement circuit 85 may deliver the drive signal as a train of pulses such that the impedance may be determined as an average value of the impedances measured for each pulse. The impedance measurement circuit 85 may sequence through impedance measurements from multiple impedance measurement electrode vectors during the train of pulses to provide an average impedance measurement for each of the multiple impedance measurement electrode vectors at the end of the train of pulses. Any technique for determining an electrical impedance between a selected electrode vector may be used in conjunction with the techniques disclosed herein for obtaining impedance measurements from multiple impedance measurement electrode vectors for use in determining a thoracic impedance estimate for monitoring a patient's fluid status.

Control circuit 80 receives the impedance measurements and determines a thoracic impedance estimate by determining an impedance of a circuit model of thoracic impedance using the multiple impedance measurements. As described in various examples below, a processor 81 of control circuit 80 may determine an equivalent impedance of a three terminal impedance circuit model representing the thoracic impedance. For instance, processor 81 may compute an impedance of the circuit model by solving for three unknown impedance elements of the circuit model using three impedance measurements received from impedance measurement circuit 85, where each of the three impedance measurements corresponds to a series and/or parallel combination of the unknown impedance elements of the circuit model. Each of three electrodes included in three impedance measurement electrode vectors may correspond to the terminals of the three terminal circuit model such that each of the impedance measurements correspond to some combination of the individual impedance elements of the circuit model. In some examples, at least three impedance measurements are obtained by impedance measurement circuit 85 and an equivalent impedance is determined according to a three-terminal wye circuit model of thoracic impedance using the three impedance measurements, as described below.

Cardiac electrical signal sensing circuit 86 (also referred to herein as “sensing circuit” 86) may be selectively coupled to electrodes 28, 30 and/or housing 15 in order to sense electrical activity of the patient's heart. Sensing circuit 86 may additionally be selectively coupled to defibrillation electrodes 24 and/or 26 for use in a sensing electrode vector together or in combination with one or more of electrodes 28, 30 and/or housing 15. Sensing circuit 86 may be enabled to selectively receive cardiac electrical signals via a pre-filter/amplifier 92 from one or more sensing electrode vectors selected from the available electrodes 24, 26, 28, 30, and housing 15 in some examples. Sensing circuit 86 may monitor one or more cardiac electrical signals for sensing cardiac events and/or producing digitized cardiac electrical signals passed to control circuit 80 for processing and analysis and/or for further transmission to external device 40 via telemetry circuit 88. For example, sensing circuit 86 may include switching circuitry for selecting which of electrodes 24, 26, 28, 30, and housing 15 are coupled to one or more sensing channels of sensing circuit 86.

Sensing circuit 86 may include an analog-to-digital converter 94, rectifier/amplifier 95, and cardiac event detector 96 configured to amplify, filter, rectify and digitize or otherwise process the cardiac electrical signal received from pre-filter/amplifier 92 to improve the signal quality for sensing cardiac electrical events, such as R-waves attendant to depolarizations of ventricular myocardium or P-waves attendant to depolarizations of atrial myocardium. Cardiac event detector 96 may include one or more sense amplifiers, threshold detectors, comparators, peak detectors, timers or other analog or digital components configured to sense cardiac events, e.g., R-waves attendant to ventricular depolarizations or P-waves attendant to atrial depolarizations. The sensed cardiac events may correspond to intrinsically depolarized cardiac tissue (no electrical pacing pulse delivered).

Sensing circuit 86 may control the amplitude of an auto-adjusting cardiac event sensing threshold over each cardiac cycle. Cardiac event detector 96 may sense a cardiac event in response to a cardiac electrical signal crossing the sensing threshold. Sensing circuit 86 may produce a cardiac sensed event signal, e.g., an atrial sensed event signal in response to a P-wave sensing threshold crossing or a ventricular sensed event signal in response to an R-wave sensing threshold crossing. The cardiac sensed event signals are passed to control circuit 80. Various sensing threshold control parameters may be used by sensing circuit 86 to set and adjust the cardiac event sensing threshold during each cardiac cycle. These sensing threshold control parameters may be stored in memory 82 and passed to sensing circuit 86 from control circuit 80 for use by hardware, firmware and/or software of control circuit 80 and/or sensing circuit 86 in controlling the amplitude of the cardiac event sensing threshold.

Control circuit 80 receives the cardiac sensed event signals from sensing circuit 86 for determining sensed event intervals, e.g., RR intervals (RRIs) and/or PP intervals (PPIs), by timing circuit 90. An RRI is the time interval between two consecutively sensed R-waves and may be determined between consecutive ventricular sensed event signals received by control circuit 80 from sensing circuit 86. A PPI is the time interval between two consecutively sensed P-waves and may be determined between consecutive atrial sensed event signals received by control circuit 80 from sensing circuit 86. Depending on programmed therapies, timing circuit 90 may trigger therapy delivery circuit 84 to generate and deliver an electrical stimulation pulse in response to a sensed event signal and/or start a pacing escape interval timer in response to a sensed event signal and restart the escape interval timer in response to the next sensed event signal. The value of the escape interval timer at the time of the next sensed event signal may be buffered in memory 82 as the sensed event interval for the associated sensed event signal. In this way, memory 82 may store a series of cardiac sensed event intervals for determining a sensed cardiac event rate.

For example, timing circuit 90 may include programmable digital counters set by a microprocessor of the control circuit 80 for controlling the basic pacing time intervals associated with various pacing modes or ATP sequences delivered by ICD 14. The microprocessor of control circuit 80 may also set the amplitude, pulse width, polarity or other characteristics of the cardiac pacing pulses, which may be based on programmed values stored in memory 82.

Timing circuit 90 may include various timers and/or counters used to control the timing of therapy delivery by therapy delivery circuit 84. In response to expiration of an escape interval timer without receiving a cardiac sensed event signal, control circuit 80 may control therapy delivery circuit 84 to generate and deliver a pacing pulse. Timing circuit 90 may additionally set time windows such as morphology template windows, morphology analysis windows or perform other timing related functions of ICD 14 including synchronizing CV/DF shocks or other therapies delivered by therapy delivery circuit 84 with sensed cardiac events.

Control circuit 80 may be configured to analyze signals received from sensing circuit 86 for detecting tachyarrhythmia. Control circuit 80 may detect tachyarrhythmia based on cardiac events sensed by sensing circuit 86 meeting tachyarrhythmia detection criteria, such as a threshold number of sensed cardiac events occurring at sensed event intervals falling in a tachyarrhythmia interval range. Control circuit 80 may be implemented in control circuit 80 as hardware, software and/or firmware that processes and analyzes signals received from sensing circuit 86 for detecting tachyarrhythmia, e.g., supraventricular tachycardia (SVT), VT and/or VF. Control circuit 80 may include comparators and counters for counting cardiac event intervals, e.g., PPIs or RRIs determined by timing circuit 90, that fall into various rate detection zones for determining an atrial rate and/or a ventricular rate or performing other rate- or interval-based assessment of cardiac sensed event signals for detecting and discriminating tachyarrhythmias.

For example, control circuit 80 may compare the RRIs determined by timing circuit 90 to one or more tachyarrhythmia detection interval zones, such as a tachycardia detection interval zone and a fibrillation detection interval zone. RRIs falling into a detection interval zone are counted by a respective VT interval counter or VF interval counter and in some cases in a combined VT/VF interval counter. In order to detect VT or VF, the respective VT or VF interval counter is required to reach a threshold “number of intervals to detect” or “NID.” The NID required to detect VT or VF may be programmed to a threshold number X VT/VF intervals out of Y consecutive RRIs. When a VT or VF interval counter reaches an NID, a ventricular tachyarrhythmia may be detected by control circuit 80. VT or VF intervals may be detected consecutively or non-consecutively out of a specified number of most recent RRIs. In some cases, a combined VT/VF interval counter may count both VT and VF intervals and detect a tachyarrhythmia episode based on the fastest intervals detected when a specified NID is reached.

Control circuit 80 may be configured to perform other signal analysis for determining if other detection criteria are satisfied before detecting VT or VF, such as R-wave morphology criteria and onset criteria. To support additional cardiac signal analyses, sensing circuit 86 may pass a digitized cardiac electrical signal, e.g., an electrocardiogram (ECG) signal, to control circuit 80 for morphology analysis performed by processor 81 for detecting and discriminating heart rhythms. A cardiac electrical signal from a selected sensing electrode vector may be passed through a filter and amplifier, provided to a multiplexer and thereafter converted to a multi-bit digital signal by ADC 94 of sensing circuit 86. Control circuit 80 may be a microprocessor-based controller that employs digital signal analysis techniques to characterize digitized signals received from sensing circuit 86 and stored in memory 82 to recognize and classify the patient's heart rhythm employing any of numerous signal processing methodologies for analyzing cardiac electrical signals and cardiac event waveforms, e.g., R-waves.

In response to detecting VT or VF, control circuit 80 may schedule a therapy and control therapy delivery circuit 84 to generate and deliver the therapy, such as ATP and/or CV/DF therapy. Therapy delivery circuit 84 is controlled by control circuit 80 to generate and deliver therapeutic electrical stimulation pulses, e.g., cardiac pacing pulses and/or CV/DF pulses. Therapy can be generated by initiating charging of high voltage capacitors included in charging circuit 72. In some examples, the high voltage therapy circuit configured to deliver CV/DF shock pulses can be controlled by control circuit 80 to deliver pacing pulses, e.g., for delivering ATP, post shock pacing pulses or ventricular pacing pulses. In other examples, therapy delivery circuit 84 may include a low voltage therapy circuit, including low voltage holding capacitors, for generating and delivering pacing pulses for a variety of pacing needs. The low voltage holding capacitors may be used in generating low voltage pulses having a voltage amplitude that is less than the capture threshold of the cardiac tissue for use in obtaining impedance threshold measurements.

It is recognized that the methods disclosed herein for obtaining impedance measurements and determining thoracic impedance estimates may be implemented in a medical device that is used for monitoring a cardiac condition without necessarily having therapy delivery capabilities or in a pacemaker that monitors cardiac electrical signals and delivers cardiac pacing therapies by therapy delivery circuit 84, e.g., CRT, without high voltage therapy capabilities such as CV/DF shock capabilities.

Control parameters utilized by control circuit 80 for sensing cardiac events and controlling therapy delivery may be programmed into memory 82 via telemetry circuit 88. Telemetry circuit 88 includes a transceiver and antenna for communicating with external device 40 (shown in FIG. 1A) using RF communication or other communication protocols as described above. Under the control of control circuit 80, telemetry circuit 88 may receive downlink telemetry from and send uplink telemetry to external device 40. Telemetry circuit 88 may transmit impedance measurements to another medical device, e.g., external device 40, for processing and analysis according to the techniques disclosed herein. In other examples, control circuit 80 may be configured to perform some or all of the analysis of impedance measurements and thoracic impedance estimates as disclosed herein and may transmit the data resulting from the analysis to external device 40. In some examples, telemetry circuit 88 is configured to transmit an alert or notification of a detected fluid status condition, e.g., detection of edema or detection of over-diuresis or dehydration. The alert or notification may be displayed to the patient or a clinician. In some examples, another medical device may receive the transmitted alert or notification and adjust a therapy delivered by the medical device receiving the transmission, e.g., by adjusting a cardiac pacing therapy such as CRT, vagal stimulation, delivery of medication such as diuretics or another therapy, to alleviate the detected fluid status condition.

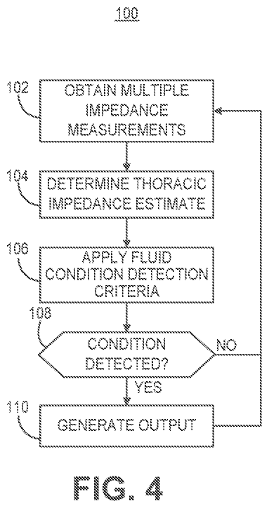

FIG. 4 is a flow chart 100 of a method that may be performed by ICD 14 for monitoring thoracic impedance according to one example. Flow chart 100 and other flow charts presented herein are described as being performed by control circuit 80, in cooperation with impedance measurement circuit 85 and/or therapy delivery circuit 84, for the sake of convenience. It is to be understood, however, that processing and analysis of impedance measurements obtained by impedance measurement circuit 85 and/or therapy delivery circuit 84 may be performed by an external processor or computer, e.g., external device processor 52 or a combination of control circuit 80 and external device processor 52.

At block 102, impedance measurement circuit 85 performs an impedance measurement from each one of multiple impedance measurement electrode vectors. The impedance measurements are passed to control circuit 80. At least two different impedance measurements may be obtained at block 102. In the illustrative examples presented herein, control circuit 80 receives at least three impedance measurements from impedance measurement circuit 85 for computing a thoracic impedance estimate. The individual impedance measurements may include impedance measurements that extend through or along only a portion of the thoracic cavity in some cases. The combination of impedance measurements, however, are used to determine an impedance of a circuit model of thoracic impedance at block 104 that represents an impedance pathway through or across the thoracic cavity and is therefore inversely correlated to the fluid in the lungs, heart and other tissues.

As described below, at least one and in some examples each impedance measurement corresponds to a series and/or parallel combination of two or more individual impedance elements of the circuit model of thoracic impedance. The number of impedance measurements may correspond to the number of individual impedance elements of the circuit model so that using the known impedance measurements, the equal number of unknown individual impedance elements of the circuit model may be solved for. The impedance of an individual element of the circuit model may be computed at block 104 as the thoracic impedance estimate in some examples. In other examples, an equivalent impedance of the circuit model, e.g., representing a network of virtual impedance elements, is computed by control circuit 80 as the thoracic impedance estimate.

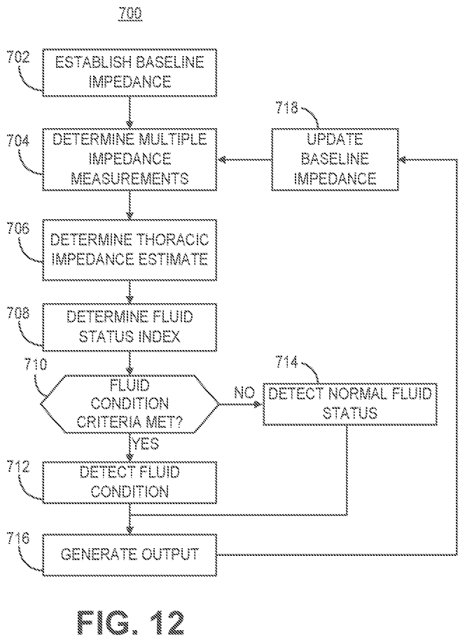

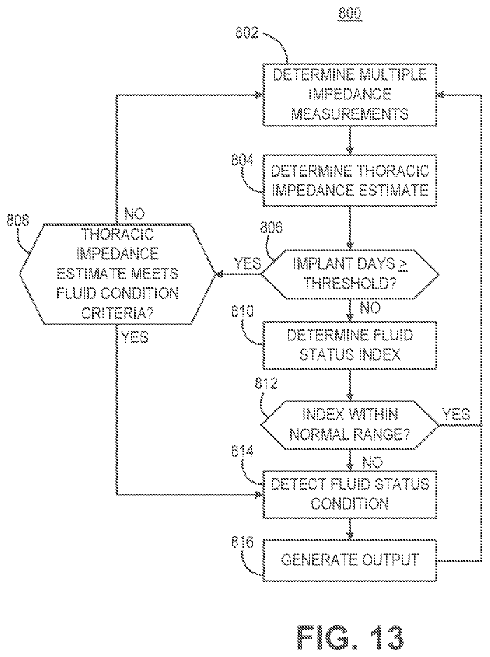

At block 106, control circuit 80 determines if the thoracic impedance estimate meets fluid condition detection criteria. As further described below, the thoracic impedance estimate may be compared to an impedance threshold or range. When the thoracic impedance estimate crosses an impedance threshold or falls outside a normal range, a fluid status condition may be detected. For example, when the thoracic impedance estimate is less than a lower impedance threshold, edema may be detected. When the thoracic impedance estimate is greater than an upper impedance threshold, over-diuresis or a dehydration condition may be detected. In other examples, the thoracic impedance estimate may be analyzed in combination with preceding thoracic impedance estimates for detecting a trend or change in a trend of thoracic impedance estimates that meets a fluid condition detection criteria. For instance, thoracic impedance estimates may be used to determine a fluid status index based on multiple, successive thoracic impedance estimates. The fluid status index may be compared to fluid condition detection criteria by control circuit 80 for detecting a fluid condition as described below in conjunction with FIGS. 12 and 13.

When the fluid condition detection criteria applied to the thoracic impedance estimate are met at block 106, control circuit 80 detects a condition, e.g., thoracic edema or over-diuresis or dehydration, at block 108. In response to detecting the fluid condition, control circuit 80 may generate an output at block 110, which may include output stored in memory 82 that is subsequently used to transmit a fluid condition detection notification and/or related data by telemetry circuit 88 and/or adjusting a therapy delivered by therapy delivery circuit 84.

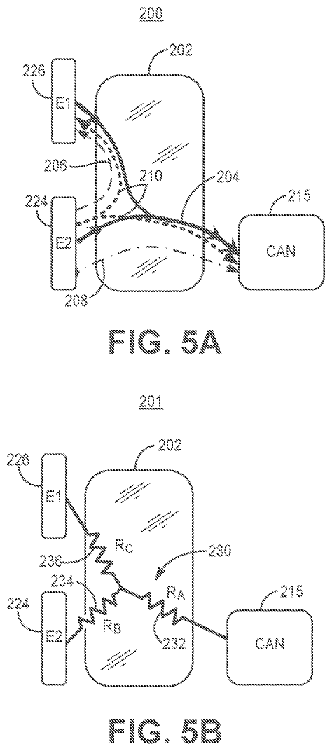

FIG. 5A is a conceptual diagram 200 of impedance measurement electrode vector pathways 206, 208 and 210 and a desired thoracic impedance pathway 204 through thoracic tissue 202 according to one example. The electrodes 224 and 226 may be carried by a lead, such as the non-transvenous lead 16 shown in FIG. 1, which may be positioned subcutaneously, submuscularly or substernally. The electrodes 224 and 226 are not required to be carried by the same lead body, however, and may be carried by two different leads coupled to the medical device performing thoracic impedance monitoring according to the techniques disclosed herein. Electrodes 224 and 226 may correspond to defibrillation coil electrodes 24 and 26 as shown in FIG. 1A but are not necessarily coil electrodes. Electrodes 224 and 226 may be coil electrodes, ring electrodes, tip electrodes or other types of electrodes or any combination thereof.

The impedance measurement circuit 85 may be configured to obtain at least three impedance measurements from three different impedance measurement electrode vectors selected from electrodes 224 and 226 and the medical device housing (sometimes referred to as a “can” electrode) 215, which may correspond to ICD housing 15 of FIG. 1A. In other examples, another electrode may be included in the three impedance measurement electrode vectors instead of or in addition to housing (“can”) 215 to provide at least three different impedance measurement vector pathways, in addition to or alternatively to any of the pathways 206, 208 and 210 shown in FIG. 5A.

Impedance measurement circuit 85 may obtain an impedance measurement between the electrodes 224 and 226 as represented by pathway 206. As described above, in some examples the impedance measurement may involve therapy delivery circuit 84 in some examples for generating a voltage or current pulse across the selected impedance measurement electrode vector, and impedance measurement circuit 85 receiving a voltage (or current) signal correlated to the tissue impedance between electrodes 224 and 226. The impedance measurement circuit 85 may obtain a second impedance measurement between the electrode 224 and housing 215 as represented by the impedance measurement vector pathway 208. A third impedance measurement may be obtained by impedance measurement circuit 85 between electrode 224 and a combination of electrode 226 and housing 215 as represented by the two branches of impedance measurement vector pathway 210. In other examples, a third impedance measurement may be between electrode 226 and housing 215 or any other combination of electrodes 224, 226 and housing 215.

The thoracic impedance pathway 204 may represent a desired thoracic impedance pathway that may be modeled as a three terminal impedance circuit model 230 as shown in FIG. 5B. As described below, the thoracic impedance pathway 204 may not be available as a selectable impedance measurement electrode vector or may yield unreliable impedance measurements during a subthreshold current or voltage drive signal due to tissue encapsulation of electrodes 226 and 224 or other factors. As such, the available impedance measurement electrode vector pathways 206, 208 and 210 may be used to obtain impedance measurements that can be used to compute an estimated impedance of the thoracic impedance pathway 204 according to a circuit model representative of the impedance pathway 204.

FIG. 5B is a diagram 201 of a three terminal impedance circuit model 230 representing the desired thoracic impedance pathway 204. Each of the electrodes 226, 224 and housing 215 may correspond to three respective terminals of the impedance circuit model 230. The impedance circuit model 230 in this example includes three individual impedance elements R_{A} 232, R_{B} 234 and R_{C} 236. The equivalent impedance presented by the three individual impedance elements R_{A} 232, R_{B} 234 and R_{C} 236 may be determined as a thoracic impedance estimate, representative of the impedance expected along the desired thoracic impedance pathway 204. Because the individual impedance elements R_{A} 123, R_{B} 234 and R_{C} 236 have unknown impedances in the circuit model 230, three impedance measurements between the three terminals of the circuit model 230, e.g., along pathways 206, 208 and 210 shown in FIG. 5A, may be used to solve for the three unknown individual impedance elements and for computing the equivalent impedance of the impedance circuit model 230 as a thoracic impedance estimate. Each of the three impedance measurements represented by the vector pathways 206, 208 and 210 correspond to a series and/or parallel combination of the unknown individual impedance elements R_{A} 232, R_{B} 234 and R_{C} 236 of circuit model 230. As such the three impedance measurements may be used to calculate the equivalent impedance of the three terminal circuit model 230 as an approximation of the thoracic impedance along pathway 204.

With continued reference to both FIGS. 5A and 5B, the first impedance measurement (Z_{E2-E1}) along pathway 206 corresponds to the series combination of the individual impedance elements R_{B} 234 and R_{C} 236, which can be expressed by Equation 1:

Z_{E2-E1}=R_{B}+R_{C} Eqn. 1:

The second impedance measurement (Z_{E2-CAN}) along pathway 208 corresponds to the series combination of the individual impedance elements R_{B} 234 and R_{A} 232, which can be expressed by Equation 2:

Z_{E2-CAN}=R_{B}+R_{A} Eqn. 2:

The third impedance measurement (Z_{E2-E1+CAN}) along the impedance measurement vector pathway 210 corresponds to the combination of the individual impedance element R_{B} 234 in series with the parallel combination of R_{A} 232 and R_{C} 236 of circuit model 230, which can be expressed by Equation 3:

Z_{E2-E1+CAN}=R_{B}+((R_{A}*R_{C})/(R_{A}+R_{C})) Eqn. 3:

By measuring three impedances, e.g., Z_{E1-E2}, Z_{E1-CAN}, and Z_{E1-E2+CAN}, the three unknown individual impedance elements, R_{A}, R_{B}, and R_{C}, of circuit model 230 can be solved for using Equations 1-3 given above. Using the computed values of the individual impedance elements R_{A}, R_{B}, and R_{C} of the three terminal circuit model 230, a thoracic impedance estimate may be computed by control circuit 80 based on the circuit model 230 and the measured impedances. For example, the equivalent impedance of circuit 230, represented by the desired thoracic impedance pathway 204, may be determined by control circuit 80 as a thoracic impedance estimate.

The desired transthoracic impedance pathway 210 as represented by the three terminal circuit model 230 corresponds to the equivalent impedance of the parallel combination of individual impedance elements R_{B} 234 and R_{C} 236 in series with R_{A} 232. This equivalent impedance may be computed as thoracic impedance estimate Z_{T} by control circuit 80 according to Equation 4:

Z_{T}=R_{A}+((R_{B}*R_{C})/(R_{B}+R_{C})) Eqn. 4:

By rearranging Equations 1-3 above to solve for each of R_{A}, R_{B}, and R_{C} and substituting those expressions into Equation 4, control circuit 80 may determine the thoracic impedance estimate Z_{T} from the three measured impedances Z_{E1-E2}, Z_{E1-CAN}, and Z_{E1-E2+CAN} (along pathways 206, 208 and 210) using Equation 5:

Z_{T}=Z_{E2-E1+CAN}(1+(Z_{E2-CAN}−2*(Z_{E2-E1+CAN}−SQRT((Z_{E1-CAN}−Z_{E2-E1+CAN})*(Z_{E1-E2}−Z_{E2-E1+CAN}))))/Z_{E2-E1}) Eqn. 5:

The diagram 201 of FIG. 5B is one example of an impedance circuit model that may be established as a network of multiple individual impedances extending between a defined number of terminals, where each terminal corresponds to an electrode (or medical device housing) that may be used in an impedance measurement electrode vector. At least one of the impedance measurement electrode vectors corresponds to a series and/or parallel combination of the unknown individual impedance elements of the circuit model such that the measured impedances may be used to compute a thoracic impedance estimate as an impedance of the circuit model, which may be an individual impedance element of the circuit model or an equivalent impedance of all or a subset of the individual impedance elements or a combination of both individual and/or equivalent impedances. In other examples, a different combination of impedance measurements than the examples described above as shown in FIG. 5A may be used to obtain a specified number of known impedance measurements that may be used to solve for the same number of unknown impedance elements in the impedance circuit model of thoracic impedance along a desired impedance pathway. Two, three, four or more impedance measurements, each corresponding to a different combination of impedance elements of the circuit model may be obtained by impedance measurement circuit 85 and passed to control module 80 for computing a thoracic impedance estimate based on the circuit model. The thoracic impedance pathway 204 represented by the circuit model 230 is one example of a desired thoracic impedance that may be estimated.

The example shown in FIGS. 5A and 5B is useful in determining an equivalent thoracic impedance corresponding to pathway 204 which is selected to approximate a thoracic impedance measurement between an electrode carried by a transvenous lead positioned in the patient's heart and a subcutaneously implanted ICD housing. It is to be understood however, that different impedance pathways may be defined as a desired thoracic impedance pathway between the electrodes corresponding to terminals of an impedance circuit model, and other circuit models different than the circuit model 230 shown in FIG. 5B may be defined for use in solving for a thoracic impedance estimate based on the circuit model and measured impedances. For example, a circuit model of impedance in the thoracic cavity or another body tissue or cavity may be defined as a star-like circuit having multiple impedance elements extending from a center node, such as the “Y” circuit described here, an “X” circuit, or other multi-branched star circuit having a center node and multiple terminals with an impedance element between each terminal and the center node. In other examples, the circuit model may be a loop or mesh-like circuit having multiple circuit elements joined end-to-end at terminals in a continuous loop, such as a delta, square, or hexagonal circuit model or the like. In some examples, the circuit model may include a combination of a star-like circuit and a mesh circuit. A tissue impedance pathway based on the selected circuit model may be defined as any series and/or parallel combination of multiple impedance elements of the circuit model. In some examples, the tissue impedance may be defined by a single impedance element of the circuit model that cannot be measured directly from available electrodes but can be computed based on measured impedances and the circuit model. The impedances of the circuit model impedance elements may be solved for based on a like number of measured impedances, each representing a combination of the impedance elements, so that the impedance of a selected pathway of the circuit model may be computed. The impedance pathway of interest may extend through any tissue plane or volume of interest.

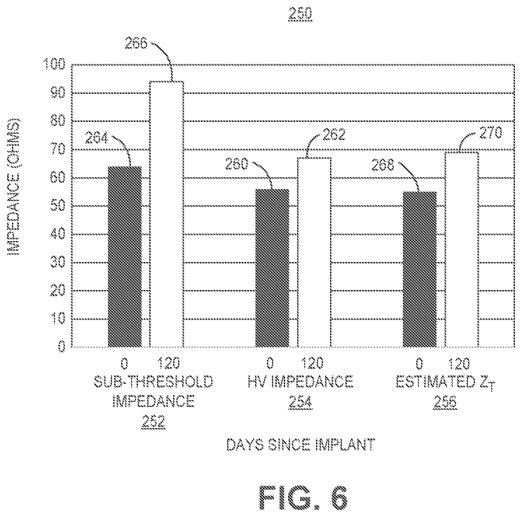

FIG. 6 is a graph 250 of impedances measured along a desired thoracic impedance pathway and thoracic impedance estimates corresponding to the desired impedance pathway computed using the techniques disclosed herein according to one example. The thoracic impedance pathway 204 shown in FIG. 5A may correspond to a high voltage CV/DF shock therapy pathway from defibrillation electrodes 24 and 26 to ICD housing 15 (as shown in FIG. 1A). ICD 14 may deliver a CV/DF shock using the defibrillation coil electrodes 24 and 26 electrically tied together as a single electrode paired with housing 15. When ICD 14 is implanted in a patient, VT or VF may be induced for delivering a test CV/DF shock to verify that the defibrillation threshold is within an acceptable limit and to enable programming of CV/DF shock delivery parameters that will promote successful termination of a spontaneous VT/VF episode when detected. A high voltage impedance measurement may be obtained during the defibrillation threshold testing. The thoracic impedance pathway 204 shown in FIG. 5A, therefore, may correspond to the HV shock pathway. During delivery of a high voltage CV/DF shock the impedance of the HV shock pathway 204 may be measured.

FIG. 6 includes a graph of this high voltage (HV) impedance 254 measured along the HV shock impedance pathway, corresponding to thoracic impedance pathway 204 of FIG. 5A, during a 20 Joule shock (greater than a defibrillation threshold energy) at 0 days since implant (HV impedance 260) and at 120 days since implant (HV impedance 262). On day 0, the HV impedance 260 is measured to be 56 ohms, and on day 120 the HV impedance 262 is measured to be 67 ohms.

FIG. 6 also includes a graph of a subthreshold impedance 252 measured along the HV shock and impedance pathway corresponding to thoracic impedance pathway 204 (from coil electrodes 24 and 26 electrically tied together as a single electrode to the housing 15) during delivery of a voltage or current pulse drive signal having a pulse energy that is less than the defibrillation threshold energy and less than a pacing capture threshold. For instance, a drive signal pulse that is approximately 0.1 milliseconds in duration and 0.02 to 0.1 milliamperes in amplitude may be injected to obtain a subthreshold impedance measurement. This subthreshold impedance 252 is measured to be 64 ohms on day 0 (impedance 264) and 94 ohms on day 120 (impedance 266).

It is noted that the 120-day HV impedance 262, measured along the impedance pathway 204 during a HV shock delivery, is much lower than the 120-day sub-threshold impedance 266 measured along the same impedance pathway 204 but during delivery of a low voltage (subthreshold) drive signal. Tissue encapsulation of the non-transvenous electrodes 24 and 26 may lead to relatively higher impedance measurements during a low voltage drive signal than during a high voltage shock delivery. As a result, impedance measured along the impedance pathway 204 shown in FIG. 5A in response to a relatively low voltage drive signal may be skewed high due to tissue encapsulation of the non-transvenous electrodes 24 and 26. This increase in impedance due to tissue encapsulation may be a confounding factor in monitoring thoracic impedance using non-transvenous electrodes for detecting a fluid status condition, e.g., edema or dehydration. Delivering high voltage drive signals along pathway 204 for measuring impedance is not practical due to pain or discomfort to the patient and high current demand on power source 98. However, greater tissue encapsulation of non-transvenous leads compared to encapsulation of leads placed in the blood stream may lead to elevated measurements of impedance measured along a desired thoracic impedance pathway when subthreshold drive signals are applied. The techniques disclosed herein provide a method for determining a reliable and more accurate thoracic impedance estimate along a desired impedance pathway from a combination of low voltage (subthreshold) impedance measurements and according to an impedance circuit model representative of the desired thoracic impedance pathway. The thoracic impedance estimate may provide a more clinically meaningful estimate of thoracic impedance for use in monitoring changes in thoracic edema and dryness.

The thoracic impedance estimate Z_{T} 256 graphed in FIG. 6 is computed according to the three terminal circuit model 230 shown in FIG. 5B using low voltage, subthreshold impedance measurements obtained along the three impedance measurement vector pathways 206, 208 and 210, described above and shown in FIG. 5A. In FIG. 6, the computed thoracic impedance estimate Z_{T} 256 is well correlated with the HV impedances 260 and 262 measured on day 0 and day 120. The thoracic impedance estimate Z_{T} 256 computed using Equation 5 above is 55 ohms on day 0 (impedance 268) similar to the 56 ohms measured for the HV impedance 260 measured on day 0. The thoracic impedance estimate Z_{T} 256 computed using Equation 5 above is 67 ohms on day 120 (impedance 270), similar to 69 ohms measured for the HV impedance measurement 262 and much less than the 94 ohms measured for the sub-threshold impedance measurement 266. The thoracic impedance estimate Z_{T} 256 is therefore a potentially more accurate and reliable estimate of thoracic impedance along a desired pathway than the impedance measured along the impedance pathway 204 during a low voltage, subthreshold drive signal. The thoracic impedance estimate Z_{T} may be less sensitive to changes in electrode impedance due to tissue encapsulation and therefore relatively more sensitive to changes in thoracic fluid content than the subthreshold impedance 252 measured along the thoracic impedance pathway 204. The elevated subthreshold impedance 266 measured at 120 days may confound thoracic impedance monitoring, potentially resulting in under detection or missed detection of edema or over detecting over-diuresis or dehydration.

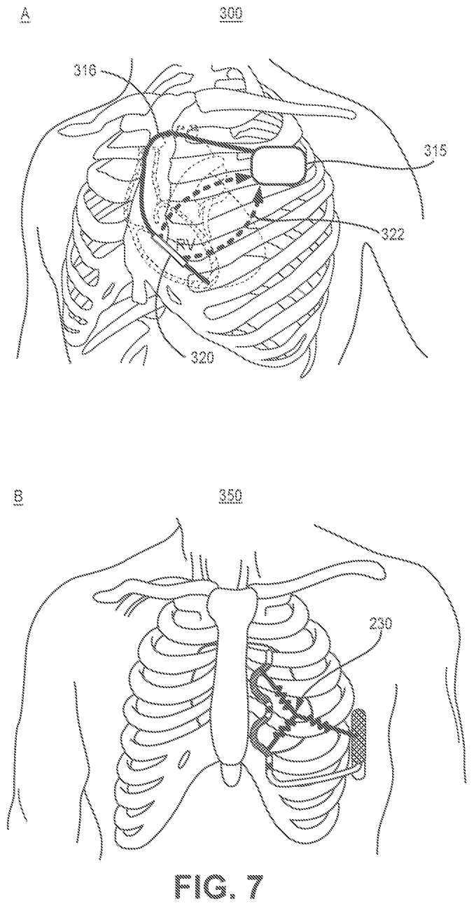

FIG. 7 is a conceptual diagram 300 of a thoracic impedance measurement obtained using a transvenous lead (in panel A) and a conceptual diagram 350 of an impedance circuit model 230 (in panel B) for determining a thoracic impedance estimate using impedance measurements obtained from a non-transvenous lead 16 according to one example. In diagram 300, a transvenous lead 316 is shown extending from an ICD housing 315 to position a defibrillation coil electrode 320 carried by lead 316 within the right ventricle (RV) of the patient's heart. An impedance measurement between the intracardiac defibrillation coil electrode 320 and the ICD housing 315 is represented by the impedance measurement vector pathway 322, which may extend through the heart and lungs of the thoracic cavity. The impedance measurement from the coil electrode 320 to the housing 315 may be used in determining a fluid status index that tracks thoracic impedance changes over time. An example of a fluid status index determined using impedance measurements from a transvenous lead is available in the OptiVol™ Fluid Status Trend Feature, Medtronic, Inc., Minneapolis, Minn., USA.

In diagram 350, the three terminal impedance circuit model 230 may be applied for determining a thoracic impedance estimate using three impedance measurements obtained using electrodes 24 and 26 and ICD housing 15, in some examples. The thoracic impedance estimate Z_{T} may be determined by control circuit 80 as described above in conjunction with FIGS. 5A and 5B, using equations 1-5. In other examples, three impedance measurements may be determined between any three combinations of impedance measurement electrode vectors selected from the available electrodes 24, 26, 28 and 30 and housing 15. The impedances measured using electrodes 24, 26, 28 and/or 30 will change over time due to encapsulation of the electrodes implanted within tissue outside the blood stream and with changes in thoracic fluid content.

While some encapsulation of the coil electrode 320 positioned in the RV may occur in the acute phase after implantation of lead 316, encapsulation of electrodes 24, 26, 28 and 30 implanted outside the heart and blood vessels may occur to a greater extent and/or over a longer post-implant phase. As a result, impedances measured using electrodes carried by a non-transvenous lead, such as lead 16, in an extra-cardiovascular ICD system may increase to a greater extent and/or over a relatively longer post-implant phase than impedances measured using transvenous or intracardiac electrodes, potentially skewing impedance measurements higher during injection of subthreshold drive signals as described above in conjunction with FIG. 6. The techniques disclosed herein for determining a thoracic impedance estimate according to a multi-terminal circuit model of thoracic impedance may at least partially compensate for the effects of greater encapsulation of the extra-cardiovascular electrodes over time and provide a thoracic impedance estimate that is better correlated to the thoracic impedance measurement pathway 322 available when transvenous, intracardiac electrodes are available. The thoracic impedance estimated using circuit model 230 may be more closely representative of a thoracic impedance measurement obtained using an intracardiac electrode or transvenous electrode, such as the intracardiac electrode 322 to housing 315, as shown in diagram 300. This type of thoracic impedance measurement represented in Panel A using an intracardiac electrode may be better known or more familiar to clinicians. The thoracic impedance estimate determined using a circuit model and impedance measurements from non-transvenous electrodes according to the techniques disclosed herein, therefore, may be more similar in magnitude over time to the impedance measurements that may be obtained using intracardiac electrodes and may more accurately represent normal, edematous, or dehydrated states of the patient.

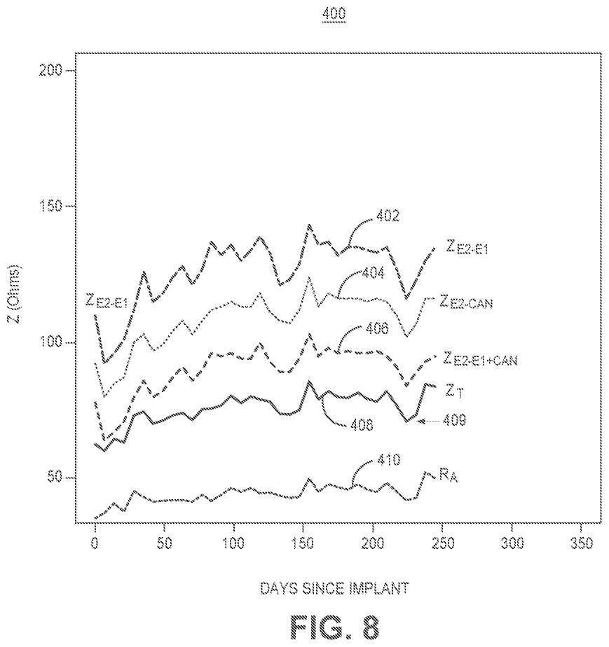

FIG. 8 is a graph 400 of measured impedances 402, 404 and 406 and computed impedance estimates 408 and 410 determined using a thoracic impedance circuit model over time according to one example. Absolute impedance values (in ohms) are plotted along the y-axis over time (in days since implant of the lead carrying the electrodes used to measure impedance) plotted along the x-axis of graph 400. The Z_{E2-E1} impedance 402 is the impedance measured between defibrillation coil electrodes 24 and 26 carried by substernal lead 16 (see, for example, FIGS. 2A-2C) in this example. The Z_{E2-CAN} impedance 404 is the impedance measured between defibrillation coil electrode 24 and the ICD housing 15. The Z_{E2-E1+CAN} impedance 406 is the impedance measured between the defibrillation coil electrode 24 and the combination of the defibrillation coil electrode 26 and ICD housing 15. The plots of impedance measurements 402, 404 and 406 may each represent an average of multiple impedance measurements taken from the given impedance measurement electrode vector over a predefined averaging time interval. For instance, impedance measurements may be received by control circuit 80 from impedance measurement circuit 85 every X minutes or hours over one day (or other averaging time interval) and averaged to obtain a daily average impedance measurement for the given impedance measurement electrode vector. In various examples, impedance is measured every 10, 20, 30, or 60 minutes and averaged to obtain the average daily impedance for each impedance measurement electrode vector. The average daily impedance may be stored in memory 82 and is represented in each of the plots 402, 404 and 406 for the respective impedance measurement electrode vector.

Control circuit 80 may be configured to control impedance measurement circuit 85 to perform impedance measurements at one or more scheduled times of day and/or at scheduled time intervals. In one example, an impedance measurement time can be scheduled to occur nightly, e.g., between midnight and 6:00 am. In other examples, impedance measurement circuit 85 may obtain an impedance measurement at one or more programmable times of day, e.g., 3:00 am, 9:00 am, 3:00 pm, and/or 9:00 pm (as illustrative non-limiting examples), with each impedance measurement stored in memory 82. The impedance measurements acquired at multiple scheduled times of day may be averaged to obtain a daily average or be used without averaging for obtaining a thoracic impedance estimate at multiple scheduled times of day.

In other examples, impedance measurements may be stored as they are obtained at a desired sampling interval, e.g., every 20 minutes, every 30 minutes, every hour, every day or other sampling interval, and stored in memory 82 without averaging. In still other examples, a different averaging interval than a one day interval may be used. For example, impedance measurements received by control circuit 80 every X minutes may be averaged to obtain an hourly impedance measurement average, a four-hour average, an eight-hour average, a twelve-hour average, or averaged over longer intervals such as a 48-hour average, a 72-hour average, or weekly average as examples. The frequency of determining an average of a series of impedance measurements from a given impedance measurement electrode vector may be tailored for individual patients, e.g., based on the severity of a heart failure condition (or heart failure class) of the patient or when a prescribed medication dosage is changed.

The plotted thoracic impedance estimate Z_{T} 408 is an equivalent impedance of the three terminal circuit model 230, which may be computed by control circuit 80 using the three measured impedances 402, 404, and 406 according to Equation 5 given above. Z_{T} 408 may be computed on a daily basis, or other scheduled frequency, using the daily averages (or other time interval averages) of the measured impedances 402, 404 and 406. In this example, a large decrease 409 in the thoracic impedance estimate Z_{T} 408, at approximately day 225, may represent an increase in edema and a worsening of the patient's heart failure condition.

The computed impedance estimate R_{A} 410 represents the estimated impedance of the individual impedance element R_{A} 232 of three terminal circuit model 230 shown in FIG. 5B. The impedance estimate R_{A} 232 may be computed using the measured impedances 402, 404 and 406 and the system of Equations 1-3 given above. R_{A} 232 corresponds to the impedance measurement Z_{E2-Can} less the series resistance R_{B} 234 of circuit model 230. Solving the system of equations 1-3 using the three known impedance measurements 402, 404 and 406, the impedance estimate R_{A} may be computed as:

R_{A}=Z_{E2-CAN}−Z_{E2-E1+CAN}+SQRT((Z_{E2-E1}−Z_{E2-E1+CAN})*(Z_{E2-E1}−Z_{E2-E1+CAN})) Eqn. 6:

The measured impedances 402, 404 and 406 tend to have an absolute impedance value that is relatively high, increasing sharply over the first 30 days or so, as the electrodes carried by non-transvenous lead 16 become encapsulated by fibrous tissue due to the body's normal foreign body response. The impedance pathway that includes R_{A} 232 in the three terminal circuit model 230 may be more representative of an impedance pathway that traverses a relatively greater portion of the thoracic cavity in the lead and electrode configurations shown in FIGS. 1A-2C. Accordingly, control circuit 80 may determine the equivalent impedance Z_{T} 408 (representing the parallel combination of R_{B} 234 and R_{C} 236 in series with R_{A} 232) or the individual impedance estimate 410 of individual impedance element R_{A} 232 as an estimation of thoracic impedance for monitoring for edema or dehydration.

The thoracic impedance estimate Z_{T} 408 determined as an equivalent impedance of circuit model 230 or the individual impedance estimate R_{A} 410 provides an absolute estimated thoracic impedance value that more closely matches the range of thoracic impedances typically measured using a transvenous, intracardiac electrode as shown in FIG. 7, panel A, and provides an impedance value that is less impacted by the greater encapsulation of the non-transvenous electrodes that may occur over time, which increases the measured impedances 402, 404 and 406 to relatively much higher values than Z_{T} 408 and R_{A} 410. The range of absolute impedance values (in ohms) of Z_{T} 408 and R_{A} 410 more closely matches the impedance (and a resulting index of patient fluid status that may be derived therefrom) that may be measured using a transvenous electrode positioned within the heart as well as the HV impedance that may be measured along the thoracic impedance pathway 204 (FIG. 5A) during a HV shock.

Clinicians working with heart failure patients may be accustomed to observing measured thoracic impedances (e.g., using a transvenous, intra-cardiac electrode) and/or associated fluid status indices based on measured thoracic impedances that correspond to the approximate magnitudes or ranges of Z_{T} 408 and R_{A} 410 as opposed to the relatively higher impedance measurements 402, 404 and 406. As such, the techniques disclosed herein provide improvements in medical device systems providing ambulatory fluid status monitoring using extra-cardiac impedance measurement electrode vectors by providing a technique for determining thoracic impedance estimates, such as equivalent thoracic impedance estimate Z_{T} 408 or individual impedance element estimate R_{A} 410, which may be determined by control circuit 80 in a non-transvenous or extra-cardiac medical device system, such as ICD system 10. In this way, the techniques disclosed herein provide continuity and consistency in the range or magnitudes of thoracic impedances determined by non-transvenous medical device systems and by transvenous medical device systems that provide fluid status monitoring.

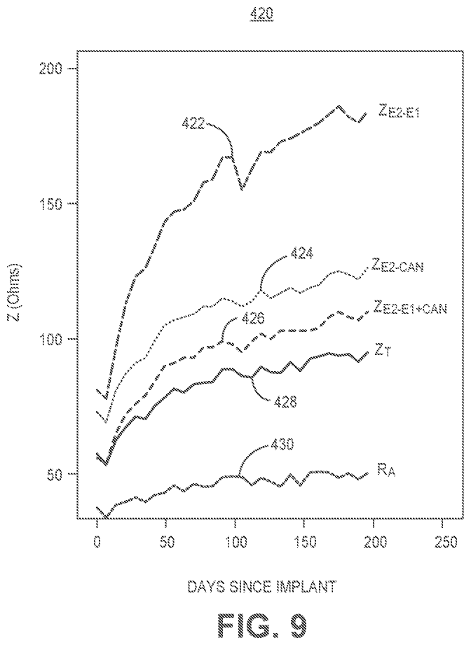

FIG. 9 is a graph 420 of measured impedances 422, 424 and 426 and determined thoracic impedance estimates 428 and 430 plotted over time according to another example. Absolute impedance values (in ohms) are plotted along the y-axis over time (in days since implant) plotted along the x-axis of graph 420. As described above, the Z_{E2-E1} impedance 422 is the measured impedance between defibrillation coil electrodes 24 and 26 carried by substernal lead 16 in this example. The Z_{E2-CAN} impedance 424 is the measured impedance between defibrillation coil electrode 24 and the ICD housing 15. The Z_{E2-E1+CAN} impedance 426 is the measured impedance between the defibrillation coil electrode 24 and the combination of the defibrillation coil electrode 26 and ICD housing 15. As described above, the plots of measured impedances 422, 424 and 426 may each represent an average of multiple impedance measurements taken from the given impedance measurement electrode vector over a given time interval. For instance, impedance measurements received by control circuit 80 from impedance measurement circuit 85 every ten minutes over a scheduled four to eight hour time period may be averaged to obtain a daily impedance measurement plotted in graph 420, as an example.

The plotted Z_{T} 428 is one example of a thoracic impedance estimate that may be computed by control circuit 80 as the equivalent impedance of a three terminal circuit model using the three measured impedances 422, 424, and 426. Z_{T} 428 may be computed on a daily basis, or other scheduled frequency, using the daily averages (or other time interval averages) of the measured impedances 422, 424 and 426. Z_{T} may be computed by control circuit 80 according to Equation 5 above, for example, and represents the equivalent impedance of the parallel combination of the circuit model impedance elements R_{B} 234 and R_{C} 236 in series with impedance element R_{A} 232 as shown in FIG. 5B.

The plotted thoracic impedance estimate R_{A} 430 represents the computed impedance of individual impedance element R_{A} 232 of three terminal circuit model 230 shown in FIG. 5B. The impedance estimate R_{A} 430 may be computed using Equation 6 above, based on the measured impedances 422, 424 and 426 and the system of Equations 1-3 given above.

The measured impedances 422, 424 and 426 tend to have an absolute impedance value that is relatively high, increasing sharply over the first 30 days or so, as the electrodes carried by non-transvenous lead 16 become encapsulated by fibrous tissue. As described above, the thoracic impedance pathway that includes R_{A} 232 in the three terminal circuit model 230 may be more representative of an impedance pathway that traverses a relatively greater portion of the thoracic cavity in the lead and electrode configurations shown in FIGS. 1A-2C. Accordingly, control circuit 80 may determine the equivalent impedance Z_{T} 428 or the individual impedance R_{A} 430 as plotted in FIG. 9 as an estimation of thoracic impedance for monitoring for edema or dehydration. As observed in graph 420, the measured impedance Z_{E2-E1} 422 between two lead based electrodes carried by a non-transvenous lead may increase significantly due to tissue encapsulation of the electrodes. The computed values of Z_{T} 428 or R_{A} 430 may provide a thoracic impedance estimate that is less sensitive to the large increase in impedance due to tissue encapsulation of the electrodes and more sensitive to actual changes in thoracic fluid content.

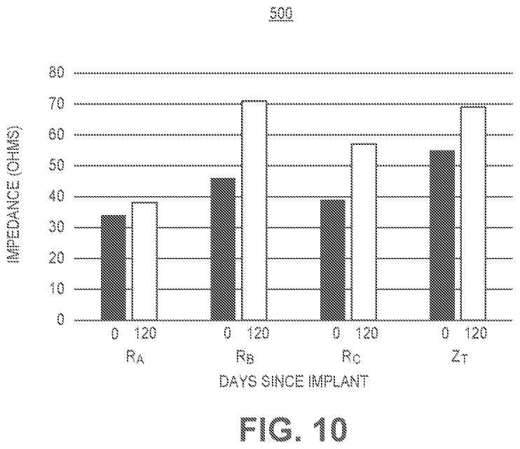

FIG. 10 is a graph 500 of computed individual impedances R_{A}, R_{B}, and R_{C} of circuit model 230 shown in FIG. 5B and the computed equivalent impedance Z_{T} determined from measured impedances at the time of implant and at 120 days after implant, according to one example. The three terminal circuit model 230 shown in FIG. 5B may include three individual impedance elements R_{A} 232, R_{B} 234, and R_{C} 236 arranged in a wye circuit model in some examples. As described above, the estimated impedances of three individual impedance elements R_{A} 232, R_{B} 234, and R_{C} 236 of a three terminal impedance circuit model may be computed using three impedance measurements and a system of three equations, where the three impedance measurements correspond to impedance measurement electrode vectors that include series and/or parallel combinations of the individual impedance elements R_{A} 232, R_{B} 234, and R_{C} 236.

For example, the system of Equations 1-3 given above may be solved using the three measured impedances Z_{E1-E2}, Z_{E1-CAN}, and Z_{E1-E2+CAN} to obtain the impedance estimates for R_{A}, R_{B}, and R_{C} shown in FIG. 10 on day 0 and day 120 (since ICD and electrode implantation). The equivalent impedance Z_{T} may be computed using Equation 5 given above and the three measured impedances Z_{E1-E2}, Z_{E1-CAN}, and Z_{E1-E2+CAN}. In some examples, R_{A} representing the individual impedance element extending through the thoracic cavity toward ICD housing 15 may be used as a thoracic impedance estimate for monitoring fluid status. Additionally or alternatively, the equivalent impedance Z_{T} of a combination of all three individual impedance elements R_{A}, R_{B}, and R_{C} may be determined and used as an estimate of thoracic impedance for monitoring a fluid status of the patient. In various examples, any combination of one, two or all three of the individual impedance elements R_{A}, R_{B}, and/or R_{C} of circuit model 230 computed using three impedance measurements may be determined and used as an estimate of thoracic impedance for monitoring a fluid status of the patient.

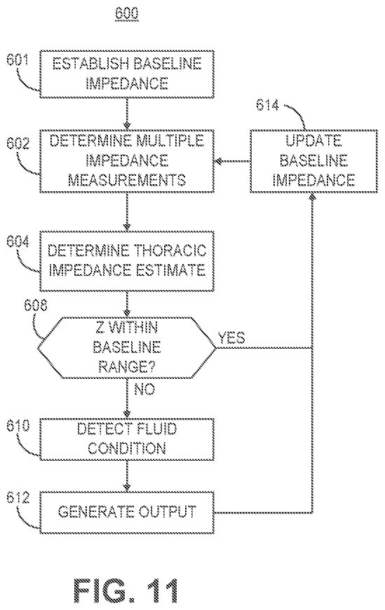

FIG. 11 is a flow chart 600 of a method for monitoring thoracic impedance according to one example. Processing and analysis of impedance measurements described in conjunction with FIG. 11 and other diagrams and flow charts presented herein are described as being performed by ICD control circuit 80 for the sake of convenience. It is to be understood, however, that impedance measurements obtained by an implantable medical device, e.g., ICD 14, may be stored in memory 82 and transmitted to another device for processing and analysis, e.g., to external device 40 of FIG. 1A for processing and analysis by processor 52.

At block 601, control circuit 80 may establish a baseline thoracic impedance. The baseline thoracic impedance may be stored in memory 82. The baseline thoracic impedance may be a predetermined reference value or normal thoracic impedance range, e.g., based on clinical data. In other examples, the baseline thoracic impedance may be determined for a given patient, e.g., by determining a long term average of thoracic impedance estimates, where each thoracic impedance estimate is computed from measured impedances according to an impedance circuit model as described above. For instance, the long term average may be determined as the average of daily calculated thoracic impedance estimates determined over a fixed or moving long-term averaging window, e.g., over one week, two weeks, one month or other selected time period.

In other examples, the baseline thoracic impedance may initially be set equal to the HV impedance measured during a test CV/DF shock delivery at the time of ICD implantation or other patient follow-up, e.g., as described above in conjunction with FIG. 6. When the thoracic impedance estimate is computed as an equivalent impedance Z_{T} of the three terminal circuit model 230, the impedance of the HV shock pathway corresponding to thoracic impedance pathway 204 (see FIG. 5A) may be used as an initial value of the baseline thoracic impedance. The baseline thoracic impedance may then be updated over a moving, averaging window using thoracic impedance estimates calculated each day (or according to another scheduled frequency) from measured impedances.

Since the measured impedances may increase during an initial healing phase due to tissue encapsulation of the electrodes, the baseline thoracic impedance may be established over a time period that begins after a delay following ICD (and electrode) implantation in some examples. Alternatively, the baseline thoracic impedance may be established using at least one moving averaging window after a post-implant delay. For example, impedance measurements for establishing the baseline thoracic impedance may begin at or within a few days after implant and continue for at least 14 days, at least 28 days, at least 35 days or other selected post-implant healing period.

At block 602, control circuit 80 controls impedance measurement circuit 85 (and/or therapy delivery circuit 86) to obtain multiple impedance measurements according to an impedance monitoring protocol. The impedance measurements may be obtained from each one of a minimum of two impedance measurement electrode vectors at block 602, but in other examples the impedance measurements are obtained from at least three different impedance measurement electrode vectors. In some examples, more than three impedance measurements may be obtained, e.g., up to six to eight impedance measurements. The number of impedance measurement electrode vectors (and the corresponding number of impedance measurements obtained at a scheduled time point) may correspond to the number of terminals or individual impedance elements of the impedance circuit model of a desired thoracic impedance pathway.

The impedance measurements are received by control circuit 80 and may be stored in memory 82. In some examples, multiple sequential impedance measurements obtained from a given impedance measurement electrode vector may be stored in memory 82 and averaged to obtain a time averaged impedance measurement for each of the impedance measurement electrode vectors for a specified time period, e.g., a daily average. Impedance measurements may be made at predetermined time intervals over a scheduled time of day, e.g., every 20 minutes over a four to eight hour period or over a five hour time period, as examples, and averaged to obtain the daily average impedance measurement for each impedance measurement electrode vector.

Control circuit 80 may compute a thoracic impedance estimate at block 604 using the impedance measurements. Determining the thoracic impedance estimate may include first determining the time averaged impedance measurements, e.g., daily average impedance measurements, from individual impedance measurements stored in memory 82. A daily thoracic impedance estimate may then be computed from the daily averaged impedance measurements. Alternatively, a thoracic impedance estimate may be determined from the individual impedance measurements stored in memory 82 each time the impedance measurements are obtained. A time averaged thoracic impedance estimate, e.g., a daily estimate, may then be determined by averaging the stored thoracic impedance estimates. Control circuit 80 may compute a thoracic impedance estimate as an equivalent impedance of an electrical circuit model of the thoracic impedance as described above. In other examples, a system of equations may be used to solve for one or more individual impedance elements of the circuit model for determining a thoracic impedance estimate. In the illustrative example given above, e.g., as shown in FIGS. 5A and 5B, the thoracic impedance estimate may be computed at block 604 as the equivalent impedance Z_{T} of the parallel combination of R_{B} 234 and R_{C} 236 in series with R_{A} 232 of the three terminal circuit model 230. Alternatively, one impedance, e.g., R_{A} 232, of the three terminal circuit model 230 may be computed as the thoracic impedance estimate using the measured impedances.

At block 608, control circuit 80 may determine if the thoracic impedance estimate is within a predetermined range of the baseline impedance. In some examples, a low impedance threshold may be defined for detecting thoracic edema. Additionally or alternatively, a high impedance threshold may be defined for detecting over-diuresis or dehydration. Therefore, a threshold range may be determined by control circuit 80 based on the baseline thoracic impedance established at block 601. The threshold range defines a “normal” thoracic impedance range for the patient. The threshold range may be centered on the baseline impedance. However, in some examples a high impedance threshold may be set at a greater absolute difference from the baseline thoracic impedance than a low impedance threshold or vice versa.

When control circuit 80 determines that a thoracic impedance estimate is within the predetermined range of the baseline thoracic impedance, control circuit 80 may return to block 602 to continue obtaining impedance measurements and determine the next thoracic impedance estimate. Control circuit 80 may determine that the fluid status of the patient is non-concerning or within a normal range for the patient. Control circuit 80 may use the current thoracic impedance estimate to update the baseline thoracic impedance at block 614 in some instances, e.g., when the baseline thoracic impedance is determined as a long term running average of the thoracic impedance estimates.

When the thoracic impedance estimate meets fluid condition detection criteria, e.g., by being outside the normal range of the baseline thoracic impedance at block 608, control circuit 80 may detect a fluid condition, e.g., edema or dehydration, at block 610. Control circuit 80 may generate an output at block 612 in response to detecting the fluid status condition. When the thoracic impedance estimate is higher than a high impedance threshold, for example, control circuit 80 may generate an output at block 612 that includes flagging the thoracic impedance estimate in memory 82 as dehydration or over-diuresis. When the thoracic impedance estimate is lower than a low impedance threshold, control circuit 80 may generate an output at block 612 that includes flagging the thoracic impedance estimate as edema in memory 82. In some examples, control circuit 80 may control telemetry circuit 88 at block 612 to transmit a notification of a fluid status condition, as flagged in memory 82, that may warrant medical attention. In some examples, the output may be generated or a notification may be transmitted at block 612 after a threshold number of consecutive or non-consecutive (e.g., X of Y) thoracic impedance estimates are consistently greater than the high impedance threshold or consistently less than the low impedance threshold thereby satisfying fluid condition detection criteria, indicating a concerning fluid status condition.

The output generated at block 612 may include transmitting impedance measurements and/or thoracic impedance estimates stored in memory 82 to enable external device 40 to receive the impedance data and generate a display of impedance measurements, thoracic impedance estimates, and/or data derived therefrom. For example, a display of daily thoracic impedance estimates graphed over time and shown relative to a threshold or threshold range may be generated by external device processor 52 for display on display unit 54, which may be part of a graphical user interface.

After detecting the fluid status condition at block 610 and generating an output at block 612, control circuit 80 may return to block 602 to continue obtaining the impedance measurements. Control circuit 80 may update the baseline thoracic impedance at block 614 using the most recent thoracic impedance estimate, e.g., by updating a long term running average thoracic impedance estimate. While not shown explicitly in FIG. 11, it is to be understood that after detecting a fluid status condition at block 610, control circuit 80 may determine when the fluid condition criteria are no longer met, e.g., when at least one or a threshold number of consecutive thoracic impedance estimates fall within the normal range of the baseline thoracic impedance. Control circuit 80 may generate an output when fluid condition criteria are no longer met or a detected fluid status condition is determined to be resolved or improved. For instance, a notification indicating normal fluid status may be transmitted by telemetry circuit 88. A return to a non-concerning fluid status may be flagged in memory 82 such that a time duration of an edematous state or a dehydrated or over-diuresed state may be determined by control circuit 88 and reported to external device 40, for example.

FIG. 12 is a flow chart 700 of a method for monitoring a fluid status of a patient according to another example. As described above in conjunction with FIG. 11, control circuit 80 may establish a baseline impedance at block 702, e.g., using any of the techniques described above. At block 704, thoracic impedance monitoring may begin by impedance measurement circuit 85 obtaining impedance measurements from each one of multiple impedance measurement electrode vectors according to a predetermined schedule or monitoring protocol. A thoracic impedance estimate is determined by control circuit 80 at block 706 from the impedance measurements according to an impedance circuit model of a desired thoracic impedance pathway using any of the example techniques described above.

Instead of comparing the thoracic impedance estimates to an impedance threshold or range, e.g., as described in conjunction with FIG. 11, control circuit 80 may determine a fluid status index at block 708 from the thoracic impedance estimate. The fluid status index may be compared to criteria for detecting a fluid condition, e.g., for detecting edema or over-diuresis or dehydration, at block 710. At block 708, control circuit 80 may determine the fluid status index based on the thoracic impedance estimate and the baseline thoracic impedance established at block 702 (which may be updated over time). Since thoracic impedance is inversely correlated to thoracic fluid content, a fluid index may be determined that is directly correlated to thoracic fluid content, so that the fluid status index increases and decreases with increasing and decreasing thoracic fluid content, respectively. In this way, recognition of a fluid condition based on observations of the fluid index may be more intuitive to interpret by a patient, clinician or caregiver.

In some examples, the fluid status index may be determined by control circuit 80 as a cumulative sum of differences between a current thoracic impedance estimate (or a relatively short term average thoracic impedance estimate) and the baseline thoracic impedance (which may be a long term average thoracic impedance estimate). As edema develops, a decrease in the thoracic impedance occurs resulting in an increasing difference between the baseline thoracic impedance and the current thoracic impedance estimate. Control circuit 80 may subtract the current thoracic impedance estimate from the baseline thoracic impedance. As edema worsens, the difference is an increasingly larger positive difference. In some examples a single difference may be determined as the fluid status index. In other examples, consecutively determined differences may be summed. When the summation of the succussive differences reaches a positive value greater than an edema threshold value, fluid condition criteria for detecting edema may be met at block 710. Edema may be detected at block 712 in response to the fluid status index crossing an upper threshold of a normal range of the fluid status index. In some examples, when the daily thoracic impedance estimate is equal to or greater than the baseline impedance (or within the normal range), control circuit 80 may reset the fluid status index to zero and restart summing consecutively determined differences between the current thoracic impedance estimate and the baseline thoracic impedance.

As edema improves, thoracic impedance increases toward the baseline thoracic impedance, resulting in decreasing differences between the baseline thoracic impedance and the current thoracic impedance estimate. As the summation of the successive differences decreases as edema resolves, control circuit 80 may determine that fluid condition criteria are no longer met at block 710. For example, when one difference or a summation of successive differences is less than a threshold value for detecting edema, control circuit 80 may determine that the fluid status of the patient has improved or that edema is no longer detected. A normal fluid status may be detected at block 714. The threshold value applied to the fluid status index for initially detecting edema may be different than the threshold value applied at block 710 to determine that edema is no longer detected to avoid frequent re-detections of a single edema episode by control circuit 80. In some examples, when control circuit 80 determines that the daily thoracic impedance estimate is equal to or greater than the baseline impedance (or within the normal range), control circuit 80 may reset the fluid status index to zero. A normal fluid status may be detected at block 714. Control circuit 80 may restart summing consecutively determined differences between the current thoracic impedance estimate and the baseline thoracic impedance.

In some cases, a patient may be instructed to increase a diuretic dosage or take other action(s) to improve a condition of edema. In other instances, a patient may be over-medicated or become dehydrated for other reasons. In a patient that is dehydrated or over-diuresed, the thoracic impedance increases resulting in a negative difference when the current thoracic impedance estimate is subtracted from the baseline thoracic impedance. When successive negative differences are summed by control circuit 80, the fluid status index determined at block 708 may become less than a negative threshold. Fluid status condition criteria for detecting dehydration or over-diuresis may be met based on the negative fluid status index in this case. The fluid status condition is detected at block 712 when the fluid status index crosses a threshold defining a normal range of the fluid status index. When the negative difference determined as the fluid status index increases above a lower normal fluid status threshold value again after detecting a dehydration or over-diuresis condition, control circuit 80 may detect a normal fluid state at block 714. In other examples, when a single thoracic impedance estimate is within a normal range of the baseline, the fluid status index may be reset to zero and normal fluid status may be detected by control circuit 80 at block 714.

In other examples, the fluid status index determined at block 708 may be the difference between the baseline thoracic impedance and the currently determined thoracic impedance estimate and the difference may be stored in memory 82, e.g., as a daily fluid status index. The daily fluid status index may be stored in a buffer, e.g., on a first in first out basis. When a threshold number of the stored differences are of the same sign (positive or negative) and are each greater than a stored fluid condition threshold difference, a fluid condition may be detected at block 712 by control circuit 80.

An output may be generated by control circuit 80 at block 716 in response to the detected fluid status condition. The output may include a status flag stored in memory 82 and/or data relating to the detected condition, including the recent impedance measurements, thoracic impedance estimates, and/or fluid status index. Telemetry circuit 88 may transmit a notification of the detected fluid status condition in response to the generated output. The transmitted notification may be received by external device 40 for use in generating a display of fluid status data. The transmitted notification may additionally or alternatively be received by another medical device capable of adjusting a therapy in response to the notification. When ICD 14 is configured to deliver CRT or other therapy for treating heart failure, therapy delivery circuit 84 may respond to the fluid condition detection output generated at block 716 by adjusting a therapy.

In some examples, control circuit 80 may advance to block 716 in response to detecting a normal fluid state at block 714, particularly when the normal fluid state is detected for the first time subsequent to detecting a fluid condition. Control circuit 80 may generate an output indicating the normal fluid state, e.g., by generating a flag stored in memory and/or a notification that is subsequently transmitted by telemetry circuit 88. In some examples, therapy delivery circuit 84 may respond to the generated output by adjusting a delivered therapy, e.g., by adjusting CRT. After generating an output corresponding to the detected fluid state, control circuit 80 may return to block 718 to update the baseline thoracic impedance and continue monitoring thoracic impedance.

FIG. 13 is a flow chart 800 of a method that may be performed by ICD 14 for monitoring thoracic impedance for detecting a fluid status condition according to another example. At block 802, impedance measurement circuit 85 obtains multiple impedance measurements, e.g., from at least three impedance measurement electrode vectors as described above. At block 804, control circuit 80 determines a thoracic impedance estimate by determining an equivalent impedance or an individual impedance element of an impedance circuit model of a desired thoracic impedance pathway using the multiple impedance measurements according to any the techniques described above.

At block 806, control circuit 80 determines if the number of days since implantation of the electrodes used for obtaining the impedance measurements, e.g., number of days since implant of ICD lead 16, has reached or exceeded a threshold number of days. The threshold number of days may be set between 21 days and 60 days, or about 30 to 35 days in some examples, and represents the acute healing phase after surgical implantation, during which the electrodes carried by lead 16 become encapsulated by tissue due to the foreign body response. Control circuit 80 may start a day counter, for example, upon connection of lead 16 to ICD 14 to track the number of days since implantation.

When the number of days since implant is less than the threshold at block 806, control circuit 80 may determine a fluid status index at block 810 for use in detecting a fluid status condition. The fluid status index may be determined as a moving, cumulative sum of differences between the current thoracic impedance estimate and a baseline thoracic impedance as described above in conjunction with FIG. 12. Control circuit 80 may compare the fluid status index to a normal fluid status index range at block 812. When the fluid status index is a positive value greater than the upper limit of the normal range, edema may be detected at block 814 and a corresponding output may be generated at block 816. When the fluid status index is a negative value less than the lower limit of the normal range, dehydration or over-diuresis may be detected at block 814 and a corresponding output generated at block 816, e.g., as a patient or clinician alert or notification that is transmitted via telemetry circuit 88. As described above, a single thoracic impedance estimate that is within a normal range of the baseline thoracic impedance may cause control circuit 80 to reset the fluid status index to zero.

When the fluid status index is within the normal range at block 812, control circuit 80 may return to block 802 to continue acquiring impedance measurements according to the thoracic impedance monitoring protocol programmed in memory 82. When the fluid status index is within the normal range, the fluid status index and/or corresponding thoracic impedance estimates and/or impedance measurements may be stored in memory 82. No notification or alert may be generated when the fluid status index is within the normal range in some examples. However, it is to be understood that in other examples, when a fluid status condition has been detected and a notification or alert has been transmitted or other output generated in response to the detected fluid status condition, control circuit 80 may transmit a notification that the fluid status condition is no longer detected in response to a subsequent fluid status index falling within the normal range at block 812.

When control circuit 80 determines that the number of days since implantation of lead 16 has reached or exceeded the threshold number of days at block 806, control circuit 80 may compare the thoracic impedance estimate directly to criteria for detecting a fluid condition at block 808. For example, the thoracic impedance estimate may be compared directly to a normal impedance range at block 808. After the initial healing phase, when impedance rises as the electrodes become encapsulated, determination of a fluid status index based on differences between the current impedance estimate and a baseline thoracic impedance may be optional. The fluid status index may account for rising impedance during the healing phase by analyzing short term and long term trends of the thoracic impedance estimate. After the initial healing phase, however, the thoracic impedance estimate may be relatively stable unless a fluid status condition is actually present or evolving, such as edema or over-diuresis. At this time, control circuit 80 may compare the thoracic impedance estimate directly to fluid condition detection criteria at block 808 without determining the fluid status index from the thoracic impedance estimate.

For example, when the thoracic impedance estimate is within a normal range at block 808, which may be based on an established baseline thoracic impedance estimate, control circuit 80 may return to block 802 to continue acquiring impedance measurements and updating the thoracic impedance estimate without detecting a fluid status condition. However, when the thoracic impedance estimate is outside the normal impedance range at block 808, control circuit 80 may detect a fluid status condition at block 814 and generate a corresponding output at block 816. In some examples, a threshold number of most recent thoracic impedance estimates may be required to be outside the normal impedance range for fluid condition detection criteria to be met at block 808. The value of the thoracic impedance estimate determined from multiple impedance measurements according to an impedance circuit model as disclosed herein is expected to provide a metric that is inversely correlated to thoracic fluid content and may be used for comparing directly to a normal impedance range for detecting a fluid status condition in some examples.

Control circuit 80 may generate an output at block 816 in response to detecting a fluid status condition, which may correspond to any of the responses to detecting a fluid status condition described above. Furthermore, as described above, when the thoracic impedance estimate returns to a normal range after being outside the normal range, or more generally when fluid condition detection criteria are no longer met as determined at block 808 after detecting a fluid condition, control circuit 80 may generate an output in response to the fluid status returning to a normal state.

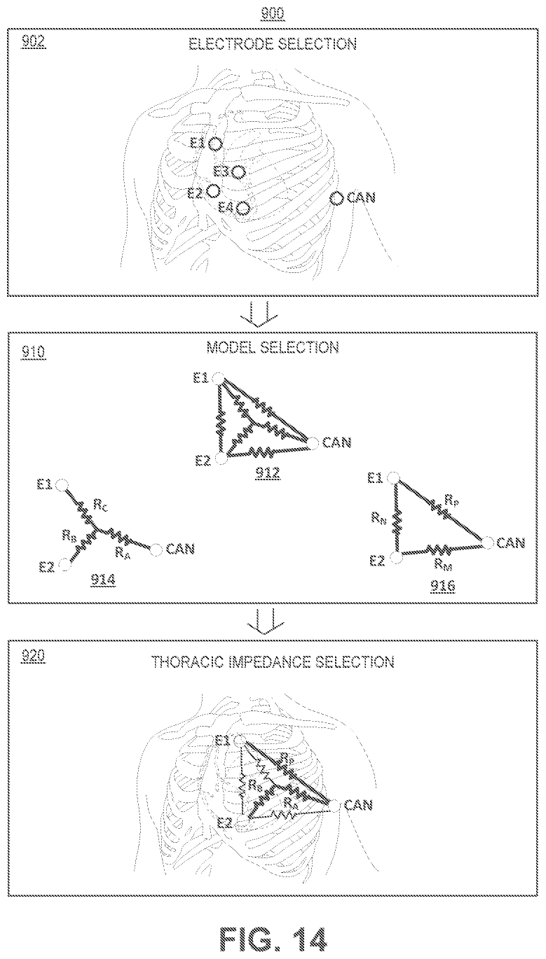

FIG. 14 is diagram 900 of a method for selecting an impedance circuit model and/or impedance elements of the circuit model for computing the impedance through a tissue or body region of interest using impedance measurements. In the illustrative examples presented above, the circuit model may be predefined, e.g., as the three terminal wye circuit model shown in FIG. 7, and the impedance determined from the circuit model may be predefined as one of the impedance elements of the model or an equivalent impedance of a combination of two or more of the impedance elements of the model. In other examples, however, the circuit model and/or the impedance element(s) of the circuit model used to estimate impedance through a body tissue or region may be selectable by a user.

The diagram 900 of FIG. 9 may represent a graphical user interface displayed by external device display unit 54 (shown in FIG. 1A) according to one example. A window 902 may display an image of approximate electrode locations of an implanted lead/electrode system. The electrodes E1, E2, E3 and E4 may correspond to approximate locations of the electrodes 24, 26, 28 and 30 carried by lead 16 (shown in FIG. 1A) and the approximate location of the housing 15 is illustrated by the CAN electrode in window 902. In some examples, a clinician may select which of the electrodes displayed in window 902 are to be used as terminals in the circuit model. The clinician may be able to add, remove or adjust the position of electrodes displayed in window 902 to indicate the electrodes that are available as terminals in a circuit model of impedance and their respective locations relative to each other the patient's anatomy. In other examples, the electrodes and approximate locations available for making impedance measurements and defining terminals of the circuit model that are displayed in window 902 may be predefined according to the medical device and lead(s) being implanted, e.g., defined according to the medical device model number or the like.

In the same window (e.g., superimposed) or a different window, display unit 54 may generate a display of the available circuit models based on the available electrodes shown or selected in window 902. For example, the defibrillation electrodes 24 and 26 (labeled E1 and E2 in window 902) may be the default impedance measurement electrodes along with the housing 15 (labeled CAN). In the illustrative example shown, these impedance measurement electrodes are shown as the three terminals of a wye circuit model 914, a delta circuit model 916, and a combined wye circuit model plus delta circuit model 912 in the circuit model selection window 910. Based on the anatomical locations of the electrodes and housing, the clinician may choose which circuit model, the combination of the wye and delta circuit models 912, wye circuit model 914 or delta circuit model 916, and/or the clinician may select which impedance elements (any subset or all impedance elements) of the selected circuit model(s) are combined for determining the tissue impedance, in this example a thoracic impedance, as shown in window 920.

In the illustrative example of FIG. 14, the clinician may select the combination of wye and delta circuit models 912 and select the series combination of impedance elements R_{B} and R_{A} in parallel with the impedance element R_{P} as the thoracic impedance that is computed using the combination of circuit models 912 and associated impedance measurements. Selections made by the user may be highlighted in the windows 902, 910 and/or 920 of the user interface by a distinguishing color, bolding, graying out non-selected electrodes, circuit models or impedance elements or other formatting techniques. In this example, the impedance of each of the six impedance elements included in the combination of circuit models 912 may be determined as a known impedance measurement (between the electrode “terminals” of one of the models such as the delta model) or solved for using the known impedance measurements and the individual circuit models.

In the illustrative example shown, if the clinician selects the impedance pathway shown in window 920, control circuit 80 may use the wye circuit model (914) to solve for the impedance elements R_{A} and R_{B} and use the delta circuit model (916) to solve for the impedance element R_{P} (or measure the impedance between E1 and CAN electrodes). Control circuit 80 may then compute the equivalent impedance of the two series impedance elements R_{A} and R_{B} determined using the wye circuit model 914 in parallel with the impedance element R_{P} from the delta circuit model 916. Thus, control circuit 80 may be configured to determine a thoracic impedance based on a combination of individual impedance elements selected from one or more circuit models for representing a desired impedance pathway through a tissue or body region. Each of the individual impedance elements of the one or more circuit models may be determined using actual impedance measurements performed between terminals of the respective circuit models as described above.

A clinician may use external device 40 to program the selected electrodes and/or locations, the selected circuit model, and/or the selected impedance elements (or impedance pathway) corresponding to the selected circuit model(s) to represent impedance through a tissue or body region for fluid status monitoring, e.g., by interacting with the windows 902, 910 and 920 generated by display unit 54. Control circuit 80 may be configured to receive the user programmed information, e.g., transmitted as a user selection signal relating to or indicating the electrode selection, circuit model selection, and/or impedance element selection, via telemetry circuit 88. Memory 82 may store the necessary equations for computing the impedances of different circuit model impedance elements and/or available impedance element combinations of each available circuit model selection, e.g., in the form of executable instructions.

In response to the user programmed selections, control circuit 80 may select the appropriate impedance measurements required for solving for the impedance elements of the selected circuit model (or combination of circuit models) and control impedance measurement circuit 85 to acquire the impedance measurements as needed according to a monitoring protocol. Control circuit 80 may select the appropriate equations and algorithms stored in memory 82 for computing the impedance of a selected impedance pathway represented by impedance elements of the selected circuit model(s) from the corresponding impedance measurements. In the example shown in FIG. 14, the selected thoracic impedance shown in window 920 may be computed by control circuit 80 using measured impedances, the selected combination of circuit models 912, and corresponding equations that may be stored in memory 82 for monitoring a fluid status of the patient according to the techniques disclosed herein.

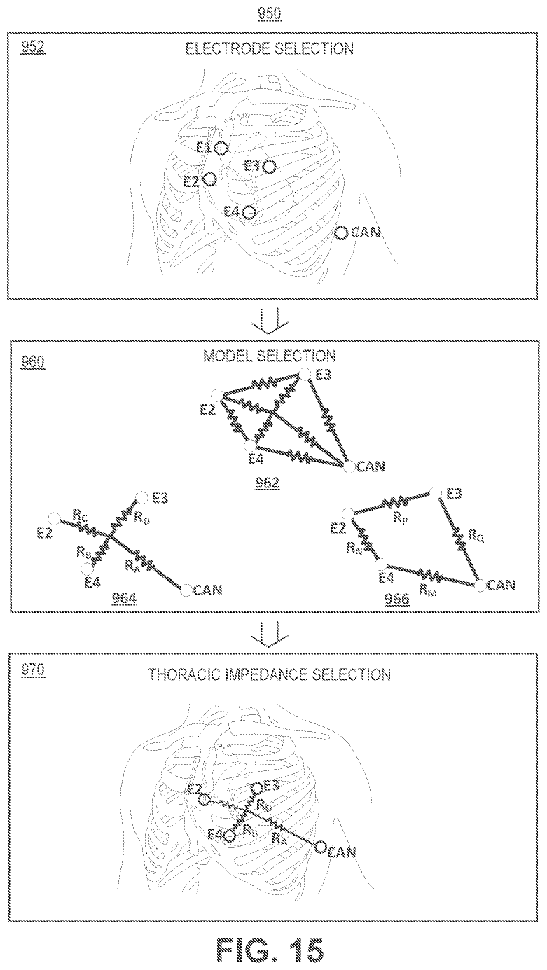

FIG. 15 is a diagram 950 of another example of a user interface that may be displayed by a display unit of an external device for enabling a user to select one or more of the impedance measurement electrodes, impedance circuit model and/or circuit model impedance elements used by the medical device for computing a tissue impedance. In this example, the electrodes E1, E2, E3 and E4 and CAN are shown at different anatomical locations in window 952. As described above, the number and location of electrodes may be displayed according to a default number and default locations based on the medical device and associated leads/electrodes being implanted. In other examples, the clinician may select the approximate locations of each available impedance measurement electrode. In this example, three electrodes E2, E3 and E4 and the CAN are selected as the impedance measurement electrodes and terminals for the circuit model, which may be a default or user selection.

The available circuit models having four terminals in this example are shown in window 960 as a star circuit model 964, a mesh circuit model 966 and a combination of the star circuit model plus the mesh circuit model 962. The impedance elements of a given circuit model (or combination of circuit models) may be selectable one, two, three or four at a time or according to predefined selectable impedance element combinations as the thoracic impedance selection. In the example shown, the user selection of the circuit model corresponds to the star circuit model 964 and the thoracic impedance is determined by the combination of the selected impedance elements R_{B} and R_{D} in series with R_{A} (as shown in window 970). The selections may be formatted, e.g., using color, bolding, graying out of non-selections, or other formatting, in the user interface in some examples.

In various examples, the circuit model may be a default model based on impedance measurement electrode selections made by a user in window 952, the circuit model may be selectable by a user in window 960 based on a default number and locations of the impedance measurement electrodes in window 952, or both the circuit model (or combination of models) and the impedance measurement electrode number and/or locations may be selectable by the user interacting with a user interface, e.g., including the windows 952 and 960, which may be displayed separately or together, e.g., in a superimposed manner.

The thoracic impedance selection (or other tissue impedance selection depending on the electrode locations and clinical application) in window 970 may be a default selection based on the user selections of electrodes and/or circuit model(s). In other examples, the user may select the circuit model impedance elements defining the thoracic impedance selection representing a desired impedance pathway, which may be based on a user selected or default electrode number and/or electrode locations and/or a user selected or default circuit model(s). As described above, memory 82 may be configured to store the equations and algorithms required to compute each selectable thoracic impedance (selected as a combination of one or more circuit model impedance elements). Control circuit 80 and impedance measurement circuit 85 may be configured to selectively obtain the corresponding impedance measurements that are required for computing the circuit model impedance elements and thoracic impedance pathway represented by one or a combination of the circuit model impedance elements.

Control circuit 80 may be configured to receive the user selections from external device 40 and operate in conjunction with memory 82 and impedance measurement circuit 85 to obtain the appropriate impedance measurements and compute the desired thoracic impedance according to the circuit model for monitoring a fluid condition. It is recognized that not all possible circuit models and/or all possible impedance elements or combinations thereof may be selectable by a user from a given number of impedance measurement electrodes and corresponding locations due to memory storage capacity, processing requirements, available impedance measurements performed by impedance measurement circuit 85, or other factors. However, at least in some examples, a user may have the flexibility of selecting one or more of the impedance measurement electrodes, the circuit model(s) and/or the impedance element(s) of the circuit model(s) that represent a desired impedance pathway through a tissue or body region that is being monitored with the understanding that these selections may be within constraints of the impedance measurements that the medical device is capable of performing and/or the number of equations and algorithms associated with different circuit models that may be stored in memory 82.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device system has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

What is claimed is:

1. A medical device comprising:
an impedance measurement circuit configured to obtain an impedance measurement between each of a plurality of impedance measurement electrode vectors;
a control circuit configured to:
determine a thoracic impedance estimate by computing an impedance of a circuit model of thoracic impedance using the impedance measurements, wherein the circuit model of thoracic impedance comprises a plurality of impedance elements extending between at least three terminals;
determine that the thoracic impedance estimate meets fluid status condition criteria;
detect a fluid status condition in response to the thoracic impedance estimate meeting the fluid status condition criteria;
generate an output in response to detecting the fluid status condition; and
a memory configured to store data relating to the thoracic impedance estimate in response to the generated output.

2. The medical device of claim 1, wherein the control circuit is further configured to determine the thoracic impedance estimate by computing an equivalent impedance of the plurality of impedance elements of the circuit model.

3. The medical device of claim 2, wherein the impedance measurement circuit is further configured to obtain each of the impedance measurements by determining an impedance measurement corresponding to a combination of at least two of the impedance elements of the circuit model.

4. The medical device of claim 2, wherein the control circuit is further configured to compute the equivalent impedance of the circuit model from the impedance measurements by computing an equivalent impedance of a wye circuit model comprising three impedance elements, wherein at least one of the impedance measurements corresponds to a series combination of at least two of the three impedance elements of the wye circuit model.

5. The medical device of claim 4, wherein the impedance measurement circuit is further configured to obtain at least one of the impedance measurements corresponding to a first impedance element of the three impedance elements of the wye circuit model in series with a parallel combination of a second impedance element and a third impedance element of the three impedance elements of the wye circuit model.

6. The medical device of claim 1, further comprising a housing enclosing the impedance measurement circuit and the control circuit, wherein:
the impedance measurement circuit is further configured to obtain the impedance measurements by determining at least:
a first impedance measurement from a first impedance measurement electrode vector of the plurality of impedance measurement electrode vectors, the first impedance measurement electrode vector being between a first electrode and a second electrode when the first and second electrodes are coupled to the impedance measurement circuit, and
a second impedance measurement from a second impedance measurement electrode vector of the plurality of impedance measurement electrode vectors, the second impedance measurement electrode vector being between the first electrode and the housing; and
the control circuit is further configured to determine the thoracic impedance estimate by determining an equivalent impedance of a three terminal circuit model using the impedance measurements, wherein the first impedance measurement corresponds to a series combination of a first impedance element and a second impedance element of the three terminal circuit model and the second impedance measurement corresponds to a series combination of the first impedance element and a third impedance element of the three terminal circuit model.

7. The medical device of claim 6, wherein the impedance measurement circuit is further configured to obtain the impedance measurements by obtaining a third impedance measurement from a third impedance measurement electrode vector of the plurality of impedance measurement electrode vectors, the third impedance measurement electrode vector being between the first electrode and a combination of the second electrode and the housing.

8. The medical device of claim 1, wherein the control circuit is further configured to determine the thoracic impedance estimate by determining an impedance of a single impedance element of the circuit model of thoracic impedance using the impedance measurements.

9. The medical device of claim 1, wherein the control circuit is further configured to determine that the thoracic impedance estimate meets the fluid status criteria by:
determining that the thoracic impedance estimate is outside a normal impedance range; and
detecting a fluid status condition in response to the thoracic impedance estimate being outside the normal impedance range.

10. The medical device of claim 1, wherein the control circuit is further configured to determine that the thoracic impedance estimate meets the fluid status criteria by:
establishing a baseline thoracic impedance;
determining a fluid status index by determining a cumulative sum of differences between a plurality of consecutively determined thoracic impedance estimates and the baseline thoracic impedance;
determining that the fluid status index crosses a threshold; and
determining that the fluid status criteria are met in response to the fluid status index crossing the threshold.

11. The medical device of claim 1, wherein the control circuit is further configured to determine the thoracic impedance estimate by computing an impedance of one of a star circuit model of the plurality of impedance elements or a mesh circuit model of the plurality of impedance elements.

12. The medical device of claim 1, further comprising a telemetry circuit configured to transmit a fluid status notification signal in response to the generated output.

13. The medical device of claim 1, wherein the impedance measurement circuit is configured to obtain the impedance measurements from a plurality of impedance measurement electrode vectors comprising at least two electrodes carried by an extra-cardiac, implantable lead.

14. The medical device of claim 1 further comprising a telemetry circuit,
wherein the control circuit is configured to:
receive a user selection signal via the telemetry circuit, the user selection signal indicating at least one of a selectable impedance measurement electrode included in the plurality of the impedance measurement electrode vectors, the circuit model of thoracic impedance, or one of the plurality of impedance elements of the circuit model; and
determine the thoracic impedance estimate by computing the impedance of the circuit model of thoracic impedance according to the user selection signal.

15. A method comprising:
obtaining an impedance measurement between each of a plurality of impedance measurement electrode vectors;
determining a thoracic impedance estimate by computing an impedance of a circuit model of thoracic impedance using the impedance measurements, the circuit model of thoracic impedance comprising a plurality of impedance elements extending between at least three terminals;
determining that the thoracic impedance estimate meets fluid status condition criteria;
detecting a fluid status condition in response to the thoracic impedance estimate meeting the fluid status condition criteria;
generating an output in response to detecting the fluid status condition; and
storing data relating to the thoracic impedance estimate in response to the generated output.

16. The method of claim 15, wherein determining the thoracic impedance estimate further comprises computing an equivalent impedance of the plurality of impedance elements of the circuit model.

17. The method of claim 16, wherein obtaining each of the impedance measurements further comprises determining an impedance measurement corresponding to a combination of at least two of the impedance elements of the circuit model.

18. The method of claim 16, wherein computing the equivalent impedance of the circuit model using the impedance measurements further comprises computing an equivalent impedance of a wye circuit model comprising three impedance elements, wherein at least one of the impedance measurements corresponds to a series combination of at least two of the three impedance elements of the wye circuit model.

19. The method of claim 18, wherein obtaining the impedance measurements further comprises obtaining an impedance measurement corresponding to a first impedance element of the three impedance elements of the wye circuit model in series with a parallel combination of a second impedance element and a third impedance element of the three impedance elements of the wye circuit model.

20. The method of claim 15, further comprising:
obtaining the impedance measurements by determining at least:
a first impedance measurement from a first impedance measurement electrode vector of the plurality of impedance measurement electrode vectors, the first impedance measurement electrode vector being between a first electrode and a second electrode, and
a second impedance measurement from a second impedance measurement electrode vector of the plurality of impedance measurement electrode vectors, the second impedance measurement electrode vector being between the first electrode and a housing of the medical device; and
determining the thoracic impedance estimate by determining an equivalent impedance of a three terminal circuit model using the impedance measurements, wherein the first impedance measurement corresponds to a series combination of a first impedance element and a second impedance element of the three terminal circuit model and the second impedance measurement corresponds to a series combination of the first impedance element and a third impedance element of the three terminal circuit model.

21. The method of claim 20, wherein obtaining the impedance measurements further comprises obtaining a third impedance measurement from a third impedance measurement electrode vector of the plurality of impedance measurement electrode vectors, the third impedance measurement electrode vector being between the first electrode and a combination of the second electrode and the housing.

22. The method of claim 15, further comprising determining the thoracic impedance estimate by determining an impedance of a single impedance element of the circuit model of thoracic impedance using the impedance measurements.

23. The method of claim 15, wherein determining that the thoracic impedance estimate meets the fluid status criteria further comprises:
determining that the thoracic impedance estimate is outside a normal impedance range; and
detecting a fluid status condition in response to the thoracic impedance estimate being outside the normal impedance range.

24. The method of claim 15, wherein determining that the thoracic impedance estimate meets the fluid status criteria further comprises:
establishing a baseline thoracic impedance;
determining a fluid status index by determining a cumulative sum of differences between a plurality of consecutively determined thoracic impedance estimates and the baseline thoracic impedance;
determining that the fluid status index crosses a threshold; and
determining that the fluid status criteria are met in response to the fluid status index crossing the threshold.

25. The method of claim 15, wherein determining the thoracic impedance estimate further comprises computing an impedance of one of a star circuit model of the plurality of impedance elements or a mesh circuit model of the plurality of impedance elements.

26. The method of claim 15 further comprising transmitting a fluid status notification signal in response to the generated output.

27. The method of claim 15, further comprising:
receiving a user selection signal indicating at least one of a selectable impedance measurement electrode included in the plurality of the impedance measurement electrode vectors, the circuit model of thoracic impedance, or one of the plurality of impedance elements of the circuit model; and
determining the thoracic impedance estimate by computing the impedance of the circuit model of thoracic impedance according to the user selection signal.

28. A non-transitory computer-readable medium storing a set of instructions which, when executed by a control circuit of a medical device, cause the device to:
obtain an impedance measurement between each of a plurality of impedance measurement electrode vectors;
determine a thoracic impedance estimate by computing an impedance of a circuit model of thoracic impedance using the impedance measurements, the circuit model of thoracic impedance comprising a plurality of impedance elements extending between at least three terminals;
determine that the thoracic impedance estimate meets fluid status condition criteria;
detect a fluid status condition in response to the thoracic impedance estimate meeting the fluid status condition criteria;
generate an output in response to detecting the fluid status condition; and
store data relating to the thoracic impedance estimate in response to the generated output.
